(19) United States
(12) Patent Application Publication
(10) Pub. No.: US 2024/0352056 A1
(43) Pub. Date: Oct. 24, 2024

(54) Improved methods for production of cyclic guanosine-monophosphate analogues

(71) Applicant: Mireca Medicines GmbH, Tübingen (DE)

(72) Inventors: Martin Bollmark, Stockholm (SE); Frank Dieter Schwede, Bremen (DE)

(73) Assignee: Mireca Medicines GmbH, Tübingen (DE)

(21) Appl. No.: 18/687,856

(22) PCT Filed: Sep. 6, 2022
(86) PCT No.: PCT/EP2022/074740

§ 371 Date: Feb. 29, 2024

(30) Foreign Application Priority Data
Sep. 6, 2021 (EP) 21195043.1

(51) Int. Cl. C07H 19/213; C07H 1/02

(52) U.S. Cl. CPC C07H 19/213; C07H 1/02

(57) ABSTRACT

The present invention relates to a method for preparing cyclic guanosine-3′, 5′-monophosphate analogues. The invention also relates to the new cyclic guanosine-monophosphate analogues and intermediates obtained by the method.

### FIELD OF THE INVENTION

The present invention relates to a method for preparing cyclic guanosine-3′, 5′-monophosphate analogues. The invention also relates to the new cyclic guanosine-monophosphate analogues and intermediates obtained by the method.

### BACKGROUND ART

Retinitis pigmentosa (RP) is a group of severely disabling inherited neurodegenerative diseases. Typically, rod photoreceptor cells—permitting vision under dim light conditions—degenerate first during the course of the disease. Subsequently, the loss of rods triggers a secondary degeneration of cone photoreceptor cells, the source of high-resolution colour vision in daylight, eventually leading to complete blindness.

Genes mutated in retinitis pigmentosa are usually associated with photoreceptor function, but there are also such that relate to general cellular functions (Kennan et al. 2005, Trends Genet. 21, 103-110). The molecule cGMP (cyclic guanosine-monophosphate) plays a direct role in the phototransduction cascade, which takes place within the photoreceptor cells when these are hit by light. In many cases, retinitis pigmentosa mutations lead to an excessive accumulation of cGMP in photoreceptors (Arango-Gonzalez et al. 2014 PLoS One. 9, e112142), for instance in situations where genes for enzymes involved in photoreceptor cGMP metabolism are affected. This is the case for mutations in phosphodiesterase 6 (whose subunits are encoded by genes PDE6B, PDE6A, PDE6G and PDE6C, PDE6H for cone photoreceptors) the photoreceptor enzymes that hydrolyse cGMP to GMP. The Pde6b gene is mutated in the rd1 mouse model of retinitis pigmentosa, which has been well studied in many laboratories. In a supposed chain of events, the accumulation of cGMP in PDE6B mutant retina occurs as a direct consequence of the actual gene defect, and this may thus be seen as an early and mechanistically fundamental degeneration component. In the next step(s), the increased cGMP can be envisaged to have at least one of four targets: 1) cGMP dependent protein kinase (protein kinase G; PKG), which when activated by cGMP, will phosphorylate specific proteins, 2) cyclic nucleotide gated ion channels (CNGC), which, when activated by cGMP, allow for a cGMP controlled influx of Na⁺ and Ca²⁺, 3) phosphodiesterase (PDE), and 4) hyperpolarization-activated cyclic nucleotide-gated (HCN) channel. The first two cGMP targets are directly connected with photoreceptor degeneration (Paquet-Durand et al. 2009, J. Neurochem. 108, 796-810; Paquet-Durand et al. 2011, Hum. Mol. Genet. 20, 941-947), while the others are known cGMP targets and hence potentially involved in the degenerative process. Due to their direct connection with the early events, PKG and CNGC can be regarded as disease drivers, even though the downstream mechanisms are still not understood in great detail (Trifunovic et al. 2012, Curr. Mol. Med. 12, 598-612).

cGMP-derived PKG inhibitors, also known as cGMP analogues (such as e.g. Rp-8-Br-cGMPS) are known to offer protection of rd1 and rd2 photoreceptors both in vitro and in in vivo mouse retinitis pigmentosa models (Paquet-Durand et al., 2009).

cGMP analogues are known in the art. WO2012130829 describes boranophosphate analogues of cyclic nucleotides. WO2018/010965 describes multimeric complexes of cGMP analogues. Butt et al. (FEBS letters, 1990, 263(1): 48, DOI: 10.1016/0014-5793(90)80702-K) describe inhibition of cGMP-dependent protein kinase by (Rp)-guanosine 3′, 5′-monophosphorothioates. Therein, activation of the cGMP-dependent protein kinase and cAMP-dependent protein kinase by the diastereomers of guanosine 3′, 5′-monophosphorothioate, (Sp)-cGMPS and (Rp)-cGMPS, and 8-chloroguanosine 3′, 5′-monophosphorothioate, (Sp)-8-Cl-cGMPS and (Rp)-8-Cl-cGMPS, was investigated. The (Sp)-diastereomers bound to the cGMP-dependent protein kinase and stimulated its phosphotransferase activity. In contrast, the (Rp)-isomers bound to the enzyme without stimulation of its activity. (Rp)-cGMPS and (Rp)-8-Cl-cGMPS antagonized the activation of the cGMP-dependent protein kinase. (Rp)-cGMPS also antagonized the activation of cAMP-dependent protein kinase. In contrast, (Rp)-8-Cl-cGMPS was a weak inhibitor of the cAMP-dependent protein kinase. (Rp)-8-Cl-cGMPS appears to be a rather selective inhibitor of the cGMP-dependent protein kinase.

Synthesis of cGMP analogues is known in the art, but known protocols are only viable at laboratory scale, such as those described in Sekhar et al. (1992, Mol. Pharmacol., 42: 103-108) and Miller et al. (1973, Biochemistry 12: 5310-5319). This is because synthesis often involves laborious purification steps involving chromatography. Extensive chromatography may be required to separate different stereoisomers of the cGMP analogues, which may be in either an Sp or Rp configuration, where the Rp stereoisomer is the more potent substance.

Synthesis of cGMP analogues, for example as described for a cAMP analogue in scheme 4 on page 18 of WO 2005/123755, involves introduction of phosphorus, followed by cyclisation. As described therein, the initial phosphorylation is thought to takes place at the 5′-OH group in the sugar, while the 2′-OH and 3′-OH group were also unprotected. This intermediate is never isolated. Instead, the products are then cyclised directly at high dilution by alkali hydroxide in aqueous acetonitrile to give the diastereoisomeric nucleosides-3′, 5′-cyclic phosphorothioates cAMP in roughly a 1:1 ratio which have to be separated by chromatographic techniques. Scheme 9 on page 23 of WO 2005/123755 describes how using phosphites instead, the diastereoisomeric ratio could be favourably shifted to 2:3 (Sp/Rp). This mixture then again had to be separated, or simply used as a mixture. In any case, known reactions are at laboratory scale.

More robust synthetic pathways and production methods with improved yield are required. There is a need for production methods that avoid laborious separation techniques. There is a need for production methods with improved stereoselectivity. There is a need for intermediates having high purity. There is a need for improved separation of different stereoisomers. There is a need for synthetic protocols that allow reactions on a larger scale.

### SUMMARY OF THE INVENTION

The invention provides a method for producing a cyclic guanosine-3′, 5′-monophosphate (cGMP) analogue or a synthetic intermediate thereof, the method comprising the steps of:
i) providing a guanosine analogue of general formula (I) or a salt thereof:

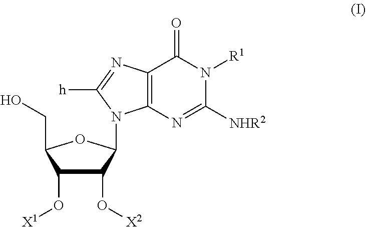

wherein h is H, halogen, or Q; X¹ and X² are each independently chosen from H or p′; p′ is in each instance independently chosen from a hydroxyl protective group; R¹ and R² are each independently chosen from H, —(CH₂)ₙ—H, —(CH₂)ₙ—C₃₋₉heterocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—; ar is in each instance independently a 5- or 6-membered aromatic or heteroaromatic ring, preferably phenyl or 2-furanyl, wherein each instance of ar is individually optionally substituted with halogen, —OH, —SH, —NH₂, —NO₂, —OCH₃, —CH₃, —CH₂CH₃, —CH(CH₃)₂, or —CF₃, and is optionally fused with a second instance of ar, preferably forming a naphthyl moiety; Q is —(CH₂)ₙ—S—(CH₂)ₙ—H, —S—(CH₂)ₙ—OH, —S—(CH₂)ₙ—NH₂, —(CH₂)ₙ—O—(CH₂)ₙ—H, —O—(CH₂)ₙ—OH, —O—(CH₂)ₙ—NH₂, —O—C(CH₃)₃, —O—CH(CH₃)₂, —(CH₂)ₙ—N(—[CH₂]ₙH)₂, —NH—(CH₂)ₙNH₂, —NH—(CH₂)ₙ—OH, —(CH₂)ₙ—Nc¹c² wherein c¹ and c² together with the N to which they are attached form a 3 to 8 membered heterocycle or wherein c¹ is H and c² is a 3 to 8 membered heterocycle, —(CH₂)ₙ—H, —N₃, —CF₃, —(CH₂)ₙ-ar, —O—(CH₂)ₙ-(ar), —NH—(CH₂)ₙ-(ar), —S—(CH₂)ₙ-(ar), —(CH₂)ₙ-amido-ar, —O—(CH₂)ₙ-amido-(ar), —NH—(CH₂)ₙ-amido-(ar), —S—(CH₂)ₙ-amido-(ar), or a linker moiety, wherein any —H may be optionally replaced by a halogen, wherein each instance of n is independently chosen from 0, 1, 2, 3, 4, 5, 6, 7, or 8;
ii) contacting the provided guanosine analogue with a phosphorous oxoacid derivative to obtain a guanosine 5′-monophosphorous oxoacid ester analogue; and
iii) isolating the obtained guanosine 5′-monophosphorous oxoacid ester analogue by crystallization. In preferred embodiments h is H or halogen or Q, preferably H or Br or Q, more preferably Br; X¹ is H and X² is p′; p′ is selected from the group consisting of methoxymethyl (MOM), tetrahydropyranyl (THP), t-butyl (tBu), allyl (all), benzyl (Bn), (tri)alkylsilyl (such as t-butyldimethylsilyl (TBDMS), triisopropylsilyl (TIPS), or t-butyldiphenylsilyl (TBDPS)), acyl (such as acetyl (Ac), pivaloyl (Pv), or benzoyl (Bz)), preferably from THP, (tri)alkylsilyl, and acyl; R¹ and R² together form —CH═C(ar)-; ar is phenyl, 4-methylphenyl, 3-thiophenyl, or 2-furanyl, preferably phenyl; and/or Q is furanyl, —CF₃, —SCH₃, —S(isopropylphenyl), —S(phenylamidomethyl), —S(halophenyl), —S(hydroxyphenyl), —S(aminophenyl), —S(nitrophenyl), —S(methoxyphenyl), —S(toluyl), —S(trifluoromethylphenyl), —Nc¹c² wherein c¹ and c² together with the N to which they are attached form a 3 to 8 membered heterocycle, —S—(CH₂)ₙ—OH, —S—(CH₂)ₙ—NH₂, —NH—(CH₂)ₙNH₂, or —NH—(CH₂)ₙOH, preferably furanyl, —CF₃, —S(4-hydroxyphenyl), or —S(4-chlorophenyl).

In preferred embodiments the guanosine analogue of general formula (I) or salt thereof used in step i) has h is Br; X¹ is H and X² is p′; p′ is triisopropylsilyl (TIPS); R¹ and R² together form —CH═C(ar)-; and ar is phenyl. In preferred embodiments the phosphorous oxoacid derivative of step ii) is a phosphorylating agent or a phosphonylating agent, preferably the phosphorous oxoacid derivative of step ii) is of general formula (P):

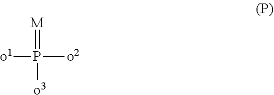

wherein M is S or O or is absent; o¹ and o² are each independently selected from halogen, —O—C₁₋₈hydrocarbon, —S—C₁₋₈hydrocarbon, —NH—C₁₋₈hydrocarbon, borano, methylborano, dimethylborano, cyanoborano, and —N(C₁₋₈hydrocarbon)₂; and o³ is H or is as defined for o¹; or o¹ and o³ together form a chiral auxiliary that is preferably a C₂₋₁₂hydrocarbon.

In preferred embodiments the guanosine 5′-monophosphorous oxoacid ester analogue obtained in step ii) is of general formula (II) or a salt thereof:

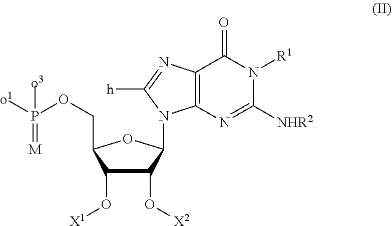

wherein o¹ and o³ are each independently —OH or as defined above; and wherein M is S or O. In preferred embodiments the guanosine analogue of general formula (I) or salt thereof is provided by the steps of: Ia) providing an unprotected guanosine analogue of general formula (pI) or a salt thereof:

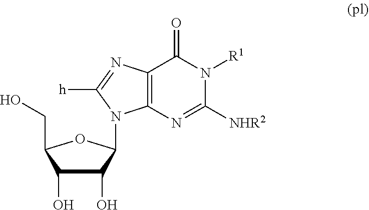

Ib) contacting the unprotected guanosine analogue with (tri)alkylsilylhalide to obtain a multiply protected guanosine analogue and optionally isolating the multiply protected guanosine analogue by crystallization; and
Ic) selectively deprotecting the multiply protected guanosine analogue to obtain the guanosine analogue of general formula (I) wherein X¹ is H and X² is p′; and
Id) optionally isolating the obtained guanosine analogue of general formula (I) wherein X¹ is H and X² is p′ by crystallization.

In preferred embodiments the method further comprises a step:
iv) cyclizing the guanosine 5′-monophosphorous oxoacid ester analogue obtained in step ii) to obtain a cyclic guanosine-3′, 5′-monophosphate (cGMP) analogue, wherein said cyclisation is preferably performed in the presence of a sterically hindered base.

Preferably the cGMP analogue is of general formula (III) or a salt thereof:

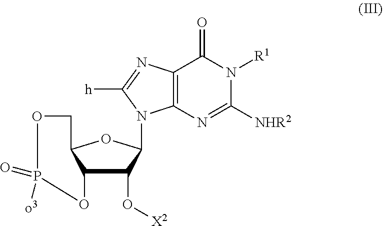

wherein preferably X² is p′ as defined above; and preferably o³ is H; and the method optionally further comprises a step: v) contacting the cGMP analogue with a sulfurizing agent to obtain a thiolated cGMP analogue of general formula (III) wherein o³ is —SH or —S—C₁₋₁₂hydrocarbon, preferably —SH. Preferably the cGMP analogue of general formula (III) is of general formula (III-Rp):

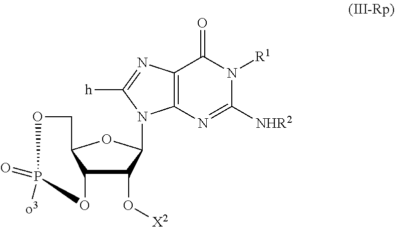

wherein preferably X² is p′ and preferably o³ is H, optionally wherein the thiolated cGMP analogue is of general formula (III-Rp), preferably X² is p′; wherein o³ is —SH or —S—C₁₋₁₂hydrocarbon, preferably —SH. Optionally X² is p′, and the method further comprises the steps of:
vi) deprotecting the hydroxyl moiety that is protected by X² to obtain a deprotected cGMP analogue; and
vii) optionally triturating the deprotected cGMP analogue; and
viii) optionally converting the deprotected cGMP analogue to a pharmaceutically acceptable salt, preferably a sodium salt.

In another aspect is provided a compound of general formula (II) or a salt thereof:

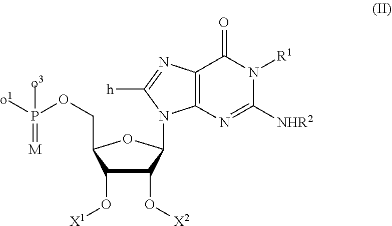

wherein h, X¹, X², R¹, and R² are as defined above; wherein o¹ and o³ are each independently —OH or as defined above, wherein preferably o³ is H or o¹ and o³ together form a chiral auxiliary that is preferably a C₂₋₁₂hydrocarbon; wherein M is S or O; wherein preferably the compound is a salt. In preferred embodiments the compound is crystalline. Preferably o³ is H. Preferably h is Br; X¹ is H and X² is p′; p′ is preferably triisopropylsilyl (TIPS); R¹ and R² together form —CH═C(ar)-; ar is phenyl; o¹ is OH; o³ is H; and M is S or O, preferably O.

### DESCRIPTION OF EMBODIMENTS

The present invention provides an improved method for synthesis of cGMP analogues. The inventors surprisingly found that a key intermediate, the guanosine 5′-monophosphorous oxoacid ester analogue formed after phosphorous is first introduced, which can also be referred to as the H-phosphonate monoester, could be crystallized. Importantly, isolation of this intermediate allowed subsequent steps to yield products having such a degree of purity that chromatographic separation is not required for cGMP synthesis using the method of this invention. In addition, use of the of the guanosine 5′-monophosphorous oxoacid ester analogue intermediate allowed more precise control over reaction conditions for subsequent cyclisation, allowing improved yield of Rp-analogues, even up to yielding Rp-analogues without requiring chromatography at any point. The method allowed synthesis at scales of as little as 50 mg to well over 100 gram. The method can be easily scaled owing to its homogeneous reaction steps, reactivity at room temperature, and lack of exotherms.

### Synthetic Method

The invention provides a method for producing a cyclic guanosine-3′, 5′-monophosphate (cGMP) analogue or a synthetic intermediate thereof, the method comprising the steps of:
i) providing a guanosine analogue of general formula (I) or a salt thereof:

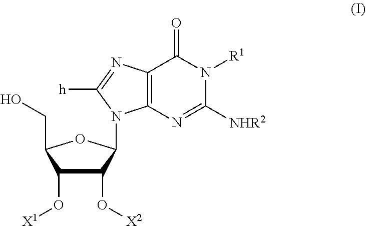

wherein:
h is H, halogen, or Q;
X¹ and X² are each independently chosen from H or p′;
p′ is in each instance independently chosen from a hydroxyl protective group;
R¹ and R² are each independently chosen from H, —(CH₂)ₙ—H, —(CH₂)ₙ—C₃₋₉heterocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—;
ar is in each instance independently a 5- or 6-membered aromatic or heteroaromatic ring, preferably phenyl or 2-furanyl, wherein each instance of ar is individually optionally substituted with halogen, —OH, —SH, —NH₂, —NO₂, —OCH₃, —CH₃, —CH₂CH₃, —CH(CH₃)₂, or —CF₃, and is optionally fused with a second instance of ar;
Q is —(CH₂)ₙ—S—(CH₂)ₙ—H, —S—(CH₂)ₙ—OH, —S—(CH₂)ₙ—NH₂, —(CH₂)ₙ—O—(CH₂)ₙ—H, —O—(CH₂)ₙ—OH, —O—(CH₂)ₙ—NH₂, —O—C(CH₃)₃, —O—CH(CH₃)₂, —(CH₂)ₙ—N(—[CH₂]ₙH)₂, —NH—(CH₂)ₙNH₂, —NH—(CH₂)ₙ—OH, —(CH₂)ₙ—Nc¹c² wherein c¹ and c² together with the N to which they are attached form a 3 to 8 membered heterocycle or wherein c¹ is H and c² is a 3 to 8 membered heterocycle, —(CH₂)ₙ—H, —N₃, —CF₃, —(CH₂)ₙ-ar, —O—(CH₂)ₙ-(ar), —NH—(CH₂)ₙ-(ar), —S—(CH₂)ₙ-(ar), —(CH₂)ₙ-amido-ar, —O—(CH₂)ₙ-amido-(ar), —NH—(CH₂)ₙ-amido-(ar), —S—(CH₂)ₙ-amido-(ar), or a linker moiety, wherein any —H may be optionally replaced by a halogen, wherein each instance of n is independently chosen from 0, 1, 2, 3, 4, 5, 6, 7, or 8;
ii) contacting the provided guanosine analogue with a phosphorous oxoacid derivative to obtain a guanosine 5′-monophosphorous oxoacid ester analogue; and
iii) isolating the obtained guanosine 5′-monophosphorous oxoacid ester analogue by crystallization.

Such a method is referred to hereinafter as the method according to the invention. The method is for the production of cGMP analogues, but is also highly suitable for producing intermediates useful in the further production of cGMP analogues. In preferred embodiments, the method is for producing cGMP analogues. In preferred embodiments, the method is for producing intermediates suitable for further conversion towards cGMP analogues. Preferred synthetic intermediates produced by the method according to the invention are compounds of general formula II, compounds of general formula III, compounds of general formula III-Rp, or salts thereof. Definitions for salts and for these compounds are provided later herein. In preferred embodiments, the intermediate is of general formula II or III-Rp. In preferred embodiments, the intermediate is of general formula III. In preferred embodiments, the intermediate is of general formula III-Rp. Compounds of general formula II are most preferred intermediates.

Preferred cGMP analogues produced by the method are described below. In general, they are governed by the target binding proteins that are to be modulated by application of the cGMP analogue. In preferred embodiments, the cGMP analogue is for activation of a cyclic guanosine-3′, 5′-monophosphate dependent protein kinase, and the preferred cGMP analogue is of general formula III wherein o³ is OH or is of general formula III-Sp wherein o³ is SH, borano, methylborano, dimethylborano, or cyanoborano. Note that said boron analogues are referred to as Rp-analogues due to lower priority of boron compared to oxygen within Cahn-Ingold-Prelog nomenclature rules.

In preferred embodiments, the cGMP analogue is for inhibition of a cyclic guanosine-3′, 5′-monophosphate dependent protein kinase, and the preferred cGMP analogue is of general formula III-Rp wherein o³ is SH, borano, methylborano, dimethylborano, cyanoborano. Said boron analogues, however, are referred to as Sp-analogues due to lower priority of boron compared to oxygen within Cahn-Ingold-Prelog nomenclature rules.

In preferred embodiments, the cGMP analogue is for activation of a cyclic guanosine-3′, 5′-monophosphate gated ion channel, and the preferred cGMP analogue is of general formula III wherein R¹ and R² do not together form —CH═C(ar)-.

In preferred embodiments the cGMP analogue is for simultaneous activation of a cyclic guanosine-3′, 5′-monophosphate dependent protein kinase and a cyclic guanosine-3′, 5′-monophosphate gated ion channel, and the preferred cGMP analogue is of general formula III-Sp wherein o³ is SH, borano, methylborano, dimethylborano, or cyanoborano, or optionally OH, and wherein R¹ and R² do not together form —CH═C(ar)-.

In preferred embodiments the cGMP analogue is for simultaneous inhibition of a cyclic guanosine-3′, 5′-monophosphate dependent protein kinase and a cyclic guanosine-3′, 5′-monophosphate gated ion channel, and the preferred cGMP analogue is of general formula III-Rp wherein o³ is S, borano, methylborano, dimethylborano, or cyanoborano, or more preferably the preferred cGMP analogue is of general formula III-Rp wherein o³ is S and wherein R¹ and R² together form —CH═C(ar)- or —(CH₂)₁₋₄C(═O)—, preferably —CH═C(ar)-, or more preferably the preferred cGMP analogue is of general formula III-Rp wherein o³ is borano, methylborano, dimethylborano, or cyanoborano and wherein R¹ and R² do not together form —CH═C(ar)- or —(CH₂)₁₋₄C(═O)—.

Further preferred examples of cyclic guanosine-3′, 5′-monophosphate analogues are:
1. 8-Bromoguanosine-3′, 5′-cyclic monophosphate (8-Br-cGMP) or its phosphorothioate (8-Br-cGMPS),
2. 8-(2, 4-dihydroxyphenylthio)guanosine-3′, 5′-cyclic monophosphate (8-o,pDHPT-cGMP) or its phosphorothioate 8-o,pDHPT-cGMPS,
3. 8-(2-aminophenylthio)guanosine-3′, 5′-cyclic monophosphate (8-APT-cGMP) or its phosphorothioate 8-APT-cGMPS,
4. 8-(4-hydroxyphenylthio)guanosine-3′, 5′-cyclic monophosphate (8-pHPT-cGMP) or its phosphorothioate 8-pHPT-cGMPS,
5. 8-(4-aminophenylthio)guanosine-3′, 5′-cyclic monophosphate (8-pAPT-cGMP) or its phosphorothioate 8-pAPT-cGMPS,
6. 8-(4-chlorophenylthio)-3-phenyl-1,N²-ethenoguanosine-3′, 5′-cyclic monophosphate (8-pCPT-PET-cGMP) or its phosphorothioate 8-pCPT-PET-cGMPS,
7. 8-(4-chlorophenylthio)guanosine-3′, 5′-cyclic monophosphate (8-pCPT-cGMP) or its phosphorothioate 8-pCPT-cGMPS,
8. 8-(2, 4-dichlorophenylthio)guanosine-3′, 5′-cyclic monophosphate (8-o,pDCIPT-cGMP) or its phosphorothioate 8-o,pDCIPT-cGMPS,
9. 8-(4-methoxyphenylthio)guanosine-3′, 5′-cyclic monophosphate (8-pMeOPT-cGMP) or its phosphorothioate 8-pMeOPT-cGMPS,
10. 8-bromo-β-phenyl-1, N²-ethenoguanosine-3′, 5′-cyclic monophosphate (8-Br-PET-cGMP) or its phosphorothioate 8-Br-PET-cGMPS,
11. 8-bromo-(2-naphthyl-1, N²-etheno)guanosine-3′, 5′-cyclic monophosphate (8-Br-(2-N)ET-cGMP) or its phosphorothioate 8-Br-(2-N)ET-cGMPS,
12. 8-(4-hydroxyphenylthio)-3-phenyl-1,N²-ethenoguanosine-3′, 5′-cyclic monophosphate (8-pHPT-PET-cGMP) or its phosphorothioate 8-pHPT-PET-cGMPS,
13. 8-(4-chlorophenylthio)-p-phenyl-1,N²-ethenoguanosine-3′, 5′-cyclic monophosphate (8-pCPT-PET-cGMP) or its phosphorothioate 8-pCPT-PET-cGMPS,
14. 2-naphthyl- 1, N²-ethenoguanosine-3′, 5′-cyclic monophosphate ((2-N)ET-cGMP) or its phosphorothioate (2-N)ET-cGMPS,
15. B3-phenyl-1, N²-ethenoguanosine-3′, 5′-cyclic monophosphate (PET-cGMP) or its phosphorothioate PET-cGMPS,
16. 4-methoxy-f3-phenyl-1,N²-ethenoguanosine-3′, 5′-monophosphate (pMeO-PET-cGMP) or its phosphorothioate pMeO-PET-cGMPS,
17. β-1, N²-acetyl-8-bromoguanosine-3′, 5′-cyclic monophosphorothioate (β-1,N²-Ac-8-Br-cGMPS) and its phosphate (β-1,N²-Ac-8-Br-cGMP),
18. 8-Bromo-b- 1, N²-butyrylguanosine-3′, 5′-cyclic monophosphorothioate (8-Br-6-1,N²-But-cGMPS) and its phosphate (8-Br-6-1,N²-But-cGMP),
19. 8-bromo-(4-methyl-p-phenyl- 1, N²-etheno)guanosine-3′, 5′-cyclic monophosphorothioate (8-Br-pMe-PET-cGMPS) and its phosphate (8-Br-pMe-PET-cGMP),
20. 8-Bromo- (3-thiophen- yl- 1, N²-etheno)guanosine-3′, 5′-cyclic monophosphorothioate (8-Br-(3-Tp)ET-cGMPS) and its phosphate (8-Br-(3-Tp)ET-cGMP),
21. 1-Benzyl- 8-bromoguanosine-3′, 5′-cyclic monophosphorothioate (1-Bn-8-Br-cGMPS) and its phosphate (1-Bn-8-Br-cGMP),
22. 8-Thioguanosine-3′, 5′-cyclic monophosphorothioate (8-T-cGMPS) and its phosphate (8-T-cGMP),
23. 8-(4-Isopropylphenylthio)guanosine-3′, 5′-cyclic monophosphorothioate (8-pIPrPT-cGMPS) and its phosphate (8-pIPrPT-cGMP),
24. 8-Phenylamidomethylthioguanosine-3′, 5′-cyclic monophosphorothioate (8-PAmdMT-cGMPS) and its phosphate (8-PAmdMT-cGMP),
25. β-phenyl- 1, N²-etheno- 8-phenylamidomethylthioguanosine-3′, 5′-cyclic monophosphorothioate (PET-8-PAmdMT-cGMPS) & phosphate (PET-8-PAmdMT-cGMP),
26. 8-(4-Isopropylphenylthio)-p-phenyl-1, N²-ethenoguanosine-3′, 5′-cyclic monophosphorothioate (8-pIPrPT-PET-cGMPS) and its phosphate (8-pIPrPT-PET-cGMP),
27. 8-(2-Aminophenylthio)- β-phenyl- 1, N²-ethenoguanosine-3′, 5′-cyclic monophosphorothioate (8-oAPT-PET-cGMPS) and its phosphate (8-oAPT-PET-cGMP),
28. β-Phenyl- 1, N²-etheno- 8-thioguanosine-3′, 5′-cyclic monophosphorothioate (PET-8-T-cGMPS) and its phosphate (PET-8-T-cGMP),
29. 8-Methylthio- β-phenyl- 1, N²-ethenoguanosine-3′, 5′-cyclic monophosphorothioate (8-MeS-PET-cGMPS) and its phosphate (8-MeS-PET-cGMP),
30. 8-Methylthio- guanosine-3′, 5′-cyclic monophosphorothioate (8-MeS-cGMPS), preferably a sodium salt, and its phosphate (8-MeS-cGMP),
31. 8-Phenylguanosine-3′, 5′-cyclic monophosphorothioate (8-Phe-cGMPS) and its phosphate (8-Phe-cGMP),
32. 8-(2-Furyl)guanosine-3′, 5′-cyclic monophosphorothioate (8-(2-Fur)-cGMPS) and its phosphate (8-(2-Fur)-cGMP),
33. 8-(4-Chlorophenyl)guanosine-3′, 5′-cyclic monophosphorothioate (8-pCP-cGMPS) and its phosphate (8-pCP-cGMP),
34. 8-Phenyl-β-phenyl- 1, N²-ethenoguanosine-3′, 5′-cyclic monophosphorothioate (8-Phe-PET-cGMPS) and its phosphate (8-Phe-PET-cGMP), and
35. 8-(4-Chlorophenyl)- β-phenyl- 1, N²-ethenoguanosine-3′, 5′-cyclic monophosphorothioate (8-pCP-PET-cGMPS) and its phosphate (8-pCP-PET-cGMP),
   and pharmaceutically acceptable salts thereof. More preferred cGMP analogues are 8-Br-cGMP, 8-Br-PET-cGMP, and 8-Br-(2-N)ET-cGMP, 8-Br-δ-1,N²-But-cGMP, 8-Br-δ-1,N²-But-cGMP, 8-Br-(3-Tp)ET-cGMP, 1-Bn-8-Br-cGMP, and their phosphorothioates, preferably their phosphorothioates. Other more preferred cGMP analogues are 8-pCPT-PET-cGMP, 8-Br-PET-cGMP, 8-pHPT-PET-cGMP, 8-pCPT-PET-cGMP, PET-cGMP, 8-Br-(2-N)ET-cGMP, (2-N)ET-cGMP, 8-Br-pMe-PET-cGMP, 8-Br-(3-Tp)ET-cGMP, PET-8-PAmdMT-cGMP, 8-pIPrPT-PET-cGMP, 8-oAPT-PET-cGMP, PET-8-T-cGMP, 8-MeS-PET-cGMP, 8-Phe-PET-cGMP, 8-pCP-PET-cGMP, and pMeO-PET-cGMP, and their phosphorothioates, preferably their phosphorothioates, more preferably 8-pCPT-PET-cGMP, 8-Br-PET-cGMP, 8-pHPT-PET-cGMP, 8-pCPT-PET-cGMP, PET-cGMP, 8-Br-pMe-PET-cGMP, PET-8-PAmdMT-cGMP, 8-piPrPT-PET-cGMP, 8-oAPT-PET-cGMP, PET-8-T-cGMP, 8-MeS-PET-cGMP, 8-Phe-PET-cGMP, 8-pCP-PET-cGMP, and pMeO-PET-cGMP, and their phosphorothioates, preferably their phosphorothioates. Other preferred cGMP analogues are β-1,N²-Ac-8-Br-cGMP and 8-Br-δ-1,N²-But-cGMP and their phosphorothioates, preferably their phosphorothioates. A most preferred cGMP analogue is 8-Br-PET-cGMP or its phosphorothioate, preferably its phosphorothioate. Another group of highly preferred cGMP analogues consists of 8-Br-PET-cGMP, 8-Br-pMe-PET-cGMP, 8-Br-(3-Tp)ET-cGMP, 8-PAmdMT-cGMP, PET-8-PAmdMT-cGMP, 8-piPrPT-PET-cGMP, and 8-oAPT-PET-cGMP, and phosphorothioates thereof, preferably Rp-isomers thereof, preferably phorphorothioates thereof, most preferably Rp-isomers of phosphorothioates thereof.

The above cGMP analogues are preferably Rp-isomers (for ease of reference, the Rp-isomer of e.g. PET-cGMP is denoted Rp-PET-cGMP, and a corresponding phosphorothioate is denoted PET-cGMPS).

### Step i) Providing a Guanosine Analogue

The first step of the method is the provision of a guanosine analogue. As used herein, guanosine itself can also be considered a guanosine analogue—it is not separately mentioned for legibility. A skilled person understands that the invention can also be practiced with guanosine, to obtain cGMP. The guanosine analogue can be synthetically prepared as part of the method, or it can be commercially obtained or otherwise sourced from elsewhere. As part of the method, phosphorous is introduced at the 5′-position of this guanosine analogue, which is later cyclized to form the cGMP analogue. It is preferred that variable moieties, such as h, R¹, and R², as later defined herein, remain constant starting from the end of step i).

The guanosine analogue (or the guanosine, as explained above) can also be a salt. In the context of the invention, a salt is preferably a pharmaceutically acceptable salt. Pharmaceutically acceptable salts are known in the art. Preferred salts are acid addition salts. Other preferred salts are base addition salts. In the context of the invention, pharmaceutically acceptable salts preferably include salts derived from inorganic bases such as Li, Na, K, Ca, Mg, Fe, Cu, Zn and Mn; salts of organic bases such as N,N′-diacetylethylenediamine, glucamine, triethylamine, choline, dicyclohexylamine, benzylamine, (tri)alkylamine, thiamine, guanidine, diethanolamine, alpha-phenylethylamine, piperidine, morpholine, pyridine, hydroxyethylpyrrolidine, hydroxyethylpiperidine, and the like. Such salts also include amino acid salts such as glycine, alanine, cystine, cysteine, lysine, arginine, phenylalanine, guanidine, etc. Such salts may include acid addition salts where appropriate, which are for example sulphates, nitrates, phosphates, perchlorates, borates, hydrohalides such as HCl or HBr salts, acetates, trifluoroacetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Preferred salts are HCl salts, formic acid salts, acetic acid salts, triethylammonium (TEAH+) salts, sodium salts, and trifluoroacetic acid salts. More preferred salts are TEAH+ salts and sodium salts. For cGMP analogues produced by the method according to the invention, preferred salts are sodium salts. For intermediates produced by the method according to the invention, or produced as part of the method according to the invention, preferred salts are TEAH+ salts.

The phrase “pharmaceutically acceptable” refers to compounds or compositions that are physiologically tolerable and do not typically produce allergic or similar untoward reactions, including but not limited to gastric upset or dizziness when administered to mammals. A skilled person can discern what is or is not pharmaceutically acceptable.

The guanosine analogue can also be a hydrate or a solvate. In the context of the invention a hydrate refers to a solvate wherein the solvent is water. The term solvate, as used herein, refers to a crystal form of a substance which contains solvent. Solvates are preferably pharmaceutically acceptable solvates and may be hydrates or may comprise other solvents of crystallization such as alcohols, ether, and the like. Accordingly, a crystal can be a hydrate or a solvate, and crystallization can yield a hydrate or a solvate.

The guanosine analogue is preferably of general formula (I) or a salt thereof:

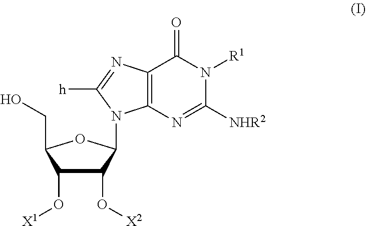

wherein:
h is H, halogen, or Q; preferably, h is H, F, Br, Cl, or Q, more preferably it is H, Br, or Q; in highly preferred embodiments, h is H; in highly preferred embodiments, h is Br; in highly preferred embodiments, h is H or Br; in highly preferred embodiments, h is Q; in most preferred embodiments, h is H or Br or Q;
X¹ and X² are each independently chosen from H or p′; preferably X¹ is H, more preferably it is H before the step wherein the cyclic phosphorous oxoacid diester is formed as described later herein; preferably, prior to formation of the 5′-phosphorous oxoacid ester, and X² is p′; most preferably, X¹ is H and X² is p′;
p′ is in each instance independently chosen from a hydroxyl protective group; Hydroxyl protective groups are known in the art and can protect oxygen atoms in hydroxyl moieties. Examples of suitable hydroxyl protective groups are extensively described in the art, e.g. by P. G. M. Wuts and T. W. Greene in Greene's Protective Groups in Organic Synthesis, Fourth Edition, 2006 (ISBN: 978-0-471-69754-1). The person skilled in the art will be able to select suitable protective groups to be used in accordance with the present invention. Examples of suitable hydroxyl protective groups are methoxymethyl (MOM), tetrahydropyranyl (THP), t-butyl (tBu), allyl (all), benzyl (Bn), (tri)alkylsilyl (such as t-butyldimethylsilyl (TBDMS), triisopropylsilyl (TIPS), or t-butyldiphenylsilyl (TBDPS), a preferred (tri)alkylsilyl is a trialkylsilyl), and acyl (such as acetyl (Ac), pivalyl (Pv), or benzoyl (Bz))., more preferably it is triisopropylsilyl (TIPS). Preferred groups for p′ are THP, (tri)alkylsilyl, and acyl. More preferred groups for p′ are THP and (tri)alkylsilyl (preferably TIPS), most preferably p′ is (tri)alkylsilyl (preferably TIPS). p′ is always attached to oxygen, and multiple instances p′ can together form a single protective group that protects more than one hydroxyl group, as is known in the art. Examples of such multivalent protective groups are acetonides and benzylidene acetals. In preferred embodiments, when two moieties protected by p′ are attached to the same carbon atom or to adjacent carbon atoms, the two instances of p′ together form a single multivalent protective group.

R¹ and R² are each independently chosen from H, —(CH₂)ₙ—H, —(CH₂)ₙ—C₃₋₉heterocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—; when n is 0, no CH₂ unit is present, and accordingly in preferred embodiments —(CH₂)ₙ—C₃₋₉heterocyclyl is C₃₋₉heterocyclyl. Heterocyclyl, in the context of the invention, is an optionally unsaturated heterocycle having 3, 4, 5, 6, 7, 8, or 9 carbon atoms. The heterocycle is optionally substituted as described for ar, and carbon atoms in the optional substitutions do not count towards C₃₋₉. Heteroatoms are preferably selected from 0, S, and N. Preferably, the heterocyclyl has at most 3 heteroatoms, more preferably at most 2, most preferably at most 1. Heterocyclyl preferably has at least 1, more preferably at least 2 heteroatoms. Preferred examples of heterocyclyl are piperidinyl, piperazinyl, morpholinyl, tetrahydrofuranyl, pyrazollyl, and pyrrolidinyl. When R¹ and R² together form —CH═C(ar)- or —(CH₂)₁₋₄C(═O)—, in preferred embodiments the ar or C(═O) is closest to where R¹ is depicted. In other preferred embodiments, ar or (C═O) is closest to where R² is depicted. Preferably, when R¹ and R² together form —CH═C(ar)- or —(CH₂)₁₋₄C(═O)—, a styrylene moiety is formed, and accordingly ar is phenyl. Most preferably, when R¹ and R² together form —CH═C(ar)-, —CH═C(phenyl)- is formed wherein phenyl is closest to R². Preferably, when R¹ and R² together form —(CH₂)₁₋₄C(═O)—, —CH₂—C(═O)— or —(CH₂)₃—C(═O)— are formed.

ar is in each instance independently a 5- or 6-membered aromatic or heteroaromatic ring, preferably phenyl or 2-furanyl, wherein each instance of ar is individually optionally substituted with halogen, —OH, —SH, —NH₂, —NO₂, —OCH₃, —CH₃, —CH₂CH₃, —CH(CH₃)₂, or —CF₃, and is optionally fused with a second instance of ar, preferably forming a naphthyl moiety; preferably no more than two instances of ar are comprised in such a fused moiety. In preferred embodiments, ar is fused to a further instance of ar. In preferred embodiments, ar is 6-membered. In preferred embodiments, ar is unsubstituted. Preferred examples of ar are phenyl, furanyl, pyridinyl, imidazolyl, tetrazolyl, triazolyl, thiophenyl, benzothiazolyl, indolyl, 4-methylphenyl, 3-thiophenyl, and naphthyl. A highly preferred embodiment of ar is 4-methylphenyl.

Q is —(CH₂)ₙ—S—(CH₂)ₙ—H, —S—(CH₂)ₙ—OH, —S—(CH₂)ₙ—NH₂, —(CH₂)ₙ—O—(CH₂)ₙ—H, —O—(CH₂)ₙ—OH, —O—(CH₂)ₙ—NH₂, —O—C(CH₃)₃, —O—CH(CH₃)₂, —(CH₂)ₙ—N(—[CH₂]ₙH)₂, —NH—(CH₂)ₙNH₂, —NH—(CH₂)ₙOH, —(CH₂)ₙ—Nc¹c² wherein c¹ and c² together with the N to which they are attached form a 3 to 8 membered heterocycle or wherein c¹ is H and c² is a 3 to 8 membered heterocycle, —(CH₂)ₙ—H, —N₃, —CF₃, —(CH₂)ₙ-ar, —O—(CH₂)ₙ-(ar), —NH—(CH₂)ₙ-(ar), —S—(CH₂)ₙ-(ar), —(CH₂)ₙ-amido-ar, —O—(CH₂)ₙ-amido-(ar), —NH—(CH₂)ₙ-amido-(ar), —S—(CH₂)ₙ-amido-(ar), or a linker moiety, wherein any —H may be optionally replaced by a halogen, wherein each instance of n is independently chosen from 0, 1, 2, 3, 4, 5, 6, 7, or 8; heterocycles have been defined above. Linkers are defined below. In preferred embodiments, Q is —(CH₂)ₙ—S—(CH₂)ₙ—H, —S—(CH₂)ₙ—OH, —S—(CH₂)ₙ—NH₂, —(CH₂)ₙ—O—(CH₂)ₙ—H, —O—(CH₂)ₙ—OH, —O—(CH₂)ₙ—NH₂, —O—C(CH₃)₃, —O—CH(CH₃)₂, —(CH₂)ₙ—N(—[CH₂]ₙH)₂, —NH—(CH₂)ₙNH₂, —NH—(CH₂)ₙOH, —(CH₂)ₙ—Nc¹c² wherein c¹ and c² together with the N to which they are attached form a 3 to 8 membered heterocycle or wherein c¹ is H and c² is a 3 to 8 membered heterocycle, —(CH₂)ₙ—H, —N₃, —CF₃, —(CH₂)ₙ-ar, —O—(CH₂)ₙ-(ar), —NH—(CH₂)ₙ-(ar), or —S—(CH₂)ₙ-(ar), wherein any —H may be optionally replaced by a halogen, wherein each instance of n is independently chosen from 0, 1, 2, 3, 4, 5, 6, 7, or 8. —(CH₂)ₙ—S—(CH₂)ₙ—H is preferably —S—(CH₂)ₙ—H or —(CH₂)ₙ—SH. For —S—(CH₂)ₙ—OH n is preferably 2, 3, 4, 5, or 6. For —S—(CH₂)ₙ—NH₂, n is preferably 2, 3, 4, 5, or 6. —(CH₂)ₙ—O—(CH₂)ₙ—H is preferably —(CH₂)ₙ—OH or —O—(CH₂)ₙ—H. For —O—(CH₂)ₙ—OH, n is preferably 2, 3, 4, 5, or 6. For —O—(CH₂)ₙ—NH₂, n is preferably 2, 3, 4, 5, or 6. —(CH₂)ₙ—N(—[CH₂]ₙH)₂ is preferably —(CH₂)ₙ—NH₂ wherein n is at least 2, or —(CH₂)ₙ—N(—[CH₂]ₙH)₂— wherein each instance of n is at least 2. For NH—(CH₂)ₙNH₂, n is preferably 2, 3, 4, 5, or 6. For —NH—(CH₂)ₙOH, n is preferably 2, 3, 4, 5, or 6. For —(CH₂)ₙ—Nc¹c² n is preferably 2, 3, 4, 5, or 6. In preferred embodiments c and C² together with the N to which they are attached form a 3 to 8 membered heterocycle, more preferably a 4 to 6 membered heterocycle, most preferably a 5 or 6 membered heterocycle. In other preferred embodiments, c¹ is H and c² is a 3 to 8 membered heterocycle, more preferably a 4, 5, or 6 membered heterocycle, most preferably a 5 or 6 membered heterocycle. For —(CH₂)ₙ—H, n is preferably 0, 1, 2, 3, 4, 5, or 6, more preferably 1, 2, 3, or 4, even more preferably 1 or 2, most preferably 1. For —(CH₂)ₙ-ar, n is preferably 0, 1, 2, 3, 4, 5, or 6, more preferably 0, 1, 2, or 3, even more preferably 0, 1 or 2, most preferably 0 or 2. For —O—(CH₂)ₙ-(ar), n is preferably at least 2. For —NH—(CH₂)ₙ-(ar), n is preferably at least 2. For —S—(CH₂)ₙ-(ar), n is preferably at least 2. Preferably, at least 1 —H may be replaced by a halogen, more preferably fluorine, and preferably at most three instances of —H are replaced by halogen; optionally, all instances of H are replaced by F. Preferably n is not 0 when this would result in directly linked heteroatoms, more preferably it is not 0 or 1. Preferably n is at most 6.

Linkers are known in the art. The structure of a linker is such that the linker can be easily chemically attached to a compound used in the invention to form a linker compound, and so that the resulting linker compound can be easily conjugated to a further substance such as a polypeptide or a surface. The choice of linker can influence the stability of such eventual conjugates when in circulation or when on a surface. Linkers may be cleavable or non-cleavable. Cleavable linkers comprise moieties that can be cleaved e.g. when exposed to lysosomal proteases or to an environment having an acidic pH. Suitable cleavable linkers are known in the art and comprise e.g. a di-, tri- or tetrapeptide, i.e., a peptide composed of two, three or four amino acid residues. Additionally, the cleavable linker may comprise a selfimmolative moiety such as an on-amino aminocarbonyl cyclization spacer, see Saari et al. J. Med. Chem., 1990, 33(1), 97-101, or a —NH—CH₂—O— moiety. Cleavage of the linker can make the compound more available to the surrounding medium. Non-cleavable linkers can still effectively release the compound according to the invention, for example after a conjugated polypeptide is degraded in the lysosome. Non-cleavable linkers include e.g. succinimidyl-4-(N-maleimidomethyl(cyclohexane)-1-carboxylate and maleimidocaproic acid and analogues thereof.

In preferred embodiments, h is H or halogen or Q, preferably H or Br or Q, more preferably Br or Q, most preferably Br;
X¹ is H and X² is p′;
p′ is selected from the group consisting of methoxymethyl (MOM), tetrahydropyranyl (THP), t-butyl (tBu), allyl (all), benzyl (Bn), (tri)alkylsilyl (such as t-butyldimethylsilyl (TBDMS), triisopropylsilyl (TIPS), or t-butyldiphenylsilyl (TBDPS)), acyl (such as acetyl (Ac), pivaloyl (Pv), or benzoyl (Bz)), preferably from THP, (tri)alkylsilyl, and acyl;
R¹ and R² together form —CH═C(ar)-;
ar is phenyl, 4-methylphenyl, 3-thiophenyl, or 2-furanyl, preferably phenyl or 2-furanyl, more preferably phenyl; and/or
Q is furanyl, —CF₃, —SCH₃, —S(isopropylphenyl), —S(phenylamidomethyl), —S(halophenyl), —S(hydroxyphenyl), —S(aminophenyl), —S(nitrophenyl), —S(methoxyphenyl), —S(toluyl), —S(trifluoromethylphenyl), —Nc¹c² wherein c¹ and c² together with the N to which they are attached form a 3 to 8 membered heterocycle, —S—(CH₂)ₙ—OH, —S—(CH₂)ₙ—NH₂, —NH—(CH₂)ₙNH₂, or —NH—(CH₂)ₙOH, preferably furanyl, —CF₃, —S(4-hydroxyphenyl), or —S(4-chlorophenyl).

Even more preferably, h is Br; X¹ is H and X² is p′; p′ is triisopropylsilyl (TIPS); R¹ and R² together form —CH═C(ar)-, wherein preferably ar is nearest to R²; and ar is phenyl or is 4-methylphenyl.

For provided GMP analogues, it is greatly preferred that X² is p′ and X¹ is H, because this facilitates chemoselective introduction of the 5′-monophosphorous oxoacid ester analogue. The inventors surprisingly found that protection at X¹ is of less influence than protection at X². Accordingly, in preferred embodiments is provided the method according to the invention, wherein X¹ is H and X² is p′, wherein the guanosine analogue of general formula (I) or salt thereof is provided by the steps of:

Ia) providing an unprotected guanosine analogue of general formula (pI) or a salt thereof:

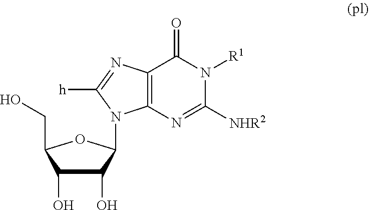

wherein h, R¹, and R² are as defined for general formula I. In this step a precursor of the guanosine analogue to be converted in step ii) is provided. This precursor is characterized in that it has H at both X¹ and X², and therefore it is referred to as an unprotected guanosine analogue.

Ib) contacting the unprotected guanosine analogue with (tri)alkylsilylhalide to obtain a multiply protected guanosine analogue and optionally isolating the multiply protected guanosine analogue by crystallization. In this step the unprotected guanosine analogue is protected using a (tri)alkylsilylhalide, leading to X² becoming a (tri)alkylsilyl protecting group, which is a preferred protecting group for p′. Additionally, the 5′-OH group can become protected during this reaction step. Surprisingly, X¹ was found to remain H. The 5′- and 2′-protected guanosine analogue can be conveniently isolated by crystallization, which is done in preferred embodiments. The reaction is preferably performed in an aprotic polar solvent such as DMF, pyridine, tetramethylurea, dimethylacetamide, or NMP, more preferably DMF or NMP. The reaction is preferably performed in the presence of a mild base, more preferably a mild organic base such as imidazole, pyridine, trialkylamine such as trimethylamine, N-methylmorpholine, or N-methylimidazole, more preferably imidazole. Progress of the reaction is preferably monitored using an analytical technique such as a chromatographic technique such as TLC or HPLC. After substantially all X² have been protected the reaction is preferably quenched, such as by addition of an excess of water, such as 10 equivalents (equiv.). The quenched reaction is preferably extracted into an aprotic organic solvent, preferably of low polarity such as toluene, esters such as isopropyl acetate and butyl acetate, ethers such as tert-butyl methyl ether, or benzene, more preferably toluene, after which it is washed with aqueous phases. The reaction product is preferably isolated by crystallization, preferably from an organic solvent that is not methanol, more preferably from an aprotic organic solvent, even more preferably from a polar one, such as ethyl acetate or propyl acetate, most preferably from isopropyl acetate. The crystallization is preferably seeded, more preferably using the desired product to be crystallized. After crystallization, which is preferably left for at least 4, more preferably at least 8, even more preferably at least 12 hours, the crystals are preferably washed and/or dried. Washing is preferably performed using the same solvent as was used for the crystallization. Drying is preferably performed in vacuum, more preferably at elevated temperature such as at about 40-80° C., preferably at about 50-70° C.

Ic) selectively deprotecting the multiply protected guanosine analogue to obtain the guanosine analogue of general formula (I) wherein X¹ is H and X² is p′. In this step the difference in reactivity of the 2′- and 5′-protected positions is used, and by using mild reaction conditions such as aqueous TFA the protected 5′-OH is selectively deprotected. This step can be performed using any combination of THE and/or water with an acid, preferably TFA, but it is preferably performed in an aprotic organic solvent, preferably in a polar one such as THE or 1,4-dioxane. Deprotection is preferably done using a strong acid, preferably TFA, preferably using about 7 to 13 such as 10 volumes of the solvent, about 1 to 3 such as 2 volumes water, and about 0.2 to 1 such as 0.45 volumes TFA. Progress of the reaction is preferably monitored using a suitable analytical technique, such as chromatography such as HPLC. Monitoring is beneficial because it can detect overdeprotection wherein the 2′-position might also deprotect. When substantially all 5′-protecting groups have been removed, the reaction is preferably quenched by addition of a base, preferably a mild nitrogen-based base such as ammonia, or a suitable amine base such as a trialkylamine, or an inorganic base such as a carbonate or bicarbonate, more preferably ammonia or an amine base as this avoids evolution of gases, most preferably ammonia as it is volume efficient. Preferably, the quenched crude reaction mixture is evaporated and resuspended in a mixture wherein fully deprotected side products are not soluble, such as in a mixture of dichloromethane or chloroform with a small amount of water (about 10-20 such as 15 volumes of the for example dichloromethane versus about 1 to 3 such as 2 volumes water), or such as acetone. The resulting thin slurry is preferentially filtered to remove possible insoluble contaminants. The organic phase of the filtrate is preferably separated after which it can be evaporated to obtain a residue. The residue is preferably triturated using a polar organic solvent, preferably an aprotic one such as acetonitrile or ethyl acetate, more preferably acetonitrile. The remaining solids are preferably washed using the same solvent as was used for trituration, after which the product is preferably dried such as vacuum dried.

Id) optionally isolating the obtained guanosine analogue of general formula (I) wherein X¹ is H and X² is p′ by crystallization or preferably trituration. Here, p′ is (tri)alkylsilyl, and the 2′-protected guanosine analogue is conveniently isolated by crystallization. Isolation is preferably performed as described above in step Ic).

When the guanosine analogue of step i) has H at both R¹ and R², the analogue can advantageously be converted into an analogue wherein R¹ and R² together form —CH═C(ar)-. To this end the analogue wherein both R¹ and R² are H is preferably dissolved in a highly polar aprotic solvent, preferably DMSO, after which about 1-3 such as 1.5 equiv. (ar)-C(═O)—CH₂Br is added. Herein, ar is as defined above, such as phenyl or furanyl, preferably phenyl. When ar is phenyl in (ar)-C(═O)—CH₂Br, the reactant is phenacylbromide. After addition of the (ar)-C(═O)—CH₂Br a strong base is added, preferably a strong organic base such as DBU or tetramethylguanidine, more preferably DBU, preferably about 1-5 such as about 2.5 equiv., preferably over a period of time such as over about 10-60 minutes such as about 30 minutes. After completion of the reaction such as after about 1h, the reaction is preferably neutralized, more preferably by addition of an acid, preferably a weak acid, more preferably an organic acid such as acetic acid. The product is preferably precipitated from the crude reaction mixture by addition of for example water such as about 10 volumes. The precipitated product is preferably isolated by filtration and is preferably washed such as with aqueous DMSO or with water, after which the solids are preferably dried such as vacuum dried. Preferably the solid is then triturated using a suitable aprotic polar organic solvent such as acetonitrile or THF, more preferably acetonitrile, after which the product is preferably dried such as vacuum dried. Vacuum drying is preferably performed at elevated temperature such as at about 70° C.

### Step ii) Formation of a Guanosine 5′-Monophosphorous Oxoacid Ester Analogue

In the second step of the method, the guanosine analogue that is provided in step i) is contacted with a phosphorous oxoacid derivative to obtain a guanosine 5′-monophosphorous oxoacid ester analogue. The phosphorous oxoacid derivative is preferably a phosphorylating agent or a phosphonylating agent, and the term phosphorous oxoacid derivative should not be so narrowly construed as to only encompass actual oxoacids—it is envisioned that thio-analogues and derivatives such as PCl₃ are also encompassed. Such agents are known in the art. In the context of this invention, the agents are for introducing phosphorous oxoacid ester at the 5′-position of the guanosine analogue to form a GMP analogue. Phosphorylating agents form an ester wherein phosphorous is in formal oxidation state P(V), such as a (HO)₂P(═O)-guanosine. Phosphonylating agents form an ester wherein phosphorous is in formal oxidation state P(Ill), such as a (HO)HP(═O)-guanosine. In preferred embodiments, the phosphorous oxoacid derivative is a phosphorylating agent. In even more preferred embodiments, the phosphorous oxoacid derivative is a phosphonylating agent.

In preferred embodiments, the phosphorous oxoacid derivative of step ii) is of general formula (P):

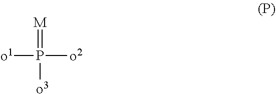

wherein:
M is S or O or is absent; in preferred embodiments, M is S or O, more preferably O; in other preferred embodiments, M is absent;
o¹ and o² are each independently selected from halogen, —O—C₁₋₈hydrocarbon, —S—C₁₋₈hydrocarbon, —NH—C₁₋₈hydrocarbon, borano, methylborano, dimethylborano, cyanoborano, and —N(C₁₋₈hydrocarbon)₂; and
o³ is H or is as defined for o¹; or o¹ and o³ together form a chiral auxiliary that is preferably a C₂₋₁₂hydrocarbon. When the compound of general formula (P) is a phosphorylating agent, o³ is not H and M is not absent. When the compound of general formula (P) is a phosphonylating agent, o³ is H, or M is absent while o¹ and o³ together form a chiral auxiliary.
C₁₋₈hydrocarbon, in this context, preferably forms a suitable leaving group. A hydrocarbon can comprise heteroatoms, and can be optionally substituted with halogen, preferably with fluorine. In preferred embodiments, o¹ represents a suitable leaving group such as halogen, —O—C₁₋₈hydrocarbon such as —O—C₆F₅, —S—C₁₋₈hydrocarbon such as —S—C₆F₅, —NH—C₁₋₈hydrocarbon, and —N(C₁₋₈hydrocarbon)₂. In preferred embodiments, o² represents a suitable leaving group such as halogen, —O—C₁₋₈hydrocarbon, —S—C₁₋₈-hydrocarbon, —NH—C₁₋₈hydrocarbon, and —N(C₁₋₈hydrocarbon)₂. In more preferred embodiments, o¹ and o² both represent a suitable leaving group. Preferably, o¹ and o² represent the same moiety. Examples of suitable leaving groups are phenol, pentafluorophenol, imidazole, triazole, diisopropylamine, and halogen such as chlorine or bromine, more preferably chlorine. It is understood that for N(C₁₋₈hydrocarbon)₂ the two hydrocarbons can together form a cyclic structure comprising the nitrogen to which they are attached, such as when imidazole is formed.

A chiral auxiliary, as is known in the art (see for example Knouse et al., 2018, Science, DOI: 10.1126/science.aau3369), is a moiety that promotes eventual cyclisation in a given stereochemical configuration, because the chiral auxiliary forms a chiral cyclic structure around the central phosphorous atom of general formula (P). An example of a chiral auxiliary, depicted here as a biradical moiety bridging between the positions where o¹ and o³ are attached, with underscores used to emphasize chiral centers, is —O—CH(C*)—C(C*)(CH₃)—S—, wherein C* and C** together with the carbon atoms to which they are attached form a cyclic structure by representing —CH₂—CH(2-propene)-CH₂—CH₂—. For compounds of general formula (P) wherein a chiral auxiliary is present, M is preferably S or O, more preferably S.

Preferred compounds of general formula (P) are diphenylphosphite, dimethylphosphite, diethylphosphite, diisopropylphosphite, dihalophosphite such as dichlorophosphite, di(pentafluorophenyl)phosphite, diphenylthiophosphite, dimethylthiophosphite, diethylthiophosphite, diisopropylthiophosphite, dihalothiophosphite such as dichlorothiophosphite, di(pentafluorophenyl)thiophosphite, di(diisopropylamino)methoxiphosphite ([(iPr)₂N]₂P[OMe]), [(iPr)₂N]₂P[OEt], Cl₂P(S)(OMe), Cl₂P(S)(OEt), Cl₂P(O)(SMe), Cl₂P(O)(SEt), (pentafluorophenyl)OP(S)=(chiral auxiliary), P(S)Cl₃, P(O)Cl₃, ClP(S)(OMe)₂, ClP(S)(OEt)₂, ClP(O)(OMe)₂, ClP(O)(OEt)₂, (pentafluorophenyl)OP(S)=(OMe)₂, (pentafluorophenyl)OP(S)=(OEt)₂, (pentafluorophenyl)OP(O)=(OMe)₂, (pentafluorophenyl)OP(O)=(OEt)₂, ClP(S)(SMe)₂, ClP(S)(SEt)₂, ClP(O)(SMe)₂, ClP(O)(SEt)₂, (pentafluorophenyl)OP(S)=(SMe)₂, (pentafluorophenyl)OP(S)=(SEt)₂, (pentafluorophenyl)OP(O)=(SMe)₂, and (pentafluorophenyl)OP(O)=(SEt)₂, more preferred are those indicated as more preferred phosphonylating or phosphorylating agents below.

Preferred phosphonylating agents are diphenylphosphite, dimethylphosphite, diethylphosphite, diisopropylphosphite, dihalophosphite such as dichlorophosphite, di(pentafluorophenyl)phosphite, diphenylthiophosphite, dimethylthiophosphite, diethylthiophosphite, diisopropylthiophosphite, dihalothiophosphite such as dichlorothiophosphite, di(pentafluorophenyl)thiophosphite, [(iPr)₂N]₂P[OMe], and [(iPr)₂N]₂P[OEt], more preferred are diphenylphosphite, diphenylthiophosphite, and [(iPr)₂N]₂P[OMe], most preferred is diphenylphosphite.

Preferred phosphorylating agents are Cl₂P(S)(OMe), Cl₂P(S)(OEt), Cl₂P(O)(SMe), Cl₂P(O)(SEt), (pentafluorophenyl)OP(S)=(chiral auxiliary), P(S)Cl₃, P(O)Cl₃, ClP(S)(OMe)₂, CIP(S)(OEt)₂, ClP(O)(OMe)₂, ClP(O)(OEt)₂, (pentafluorophenyl)OP(S)=(OMe)₂, (pentafluorophenyl)OP(S)=(OEt)₂, (pentafluorophenyl)OP(O)=(OMe)₂, (pentafluorophenyl)OP(O)=(OEt)₂, ClP(S)(SMe)₂, ClP(S)(SEt)₂, ClP(O)(SMe)₂, ClP(O)(SEt)₂, (pentafluorophenyl)OP(S)=(SMe)₂, (pentafluorophenyl)OP(S)=(SEt)₂, (pentafluorophenyl)OP(O)=(SMe)₂, and (pentafluorophenyl)OP(O)=(SEt)₂, more preferred are Cl₂P(S)(OMe), Cl₂P(O)(SMe), (pentafluorophenyl)OP(S)=(chiral auxiliary), P(S)Cl₃, P(O)Cl₃, CIP(S)(OMe)₂, ClP(O)(OMe)₂, ClP(S)(SMe)₂, and ClP(O)(SMe)₂.

Contacting of the phosphorous oxoacid derivative with the guanosine analogue is preferably performed under conditions conducive to formation of a guanosine 5′-monophosphorous oxoacid ester analogue. The contacting is preferably performed in an aprotic solvent, more preferably in an aprotic solvent of relatively low polarity such as dichloromethane or chloroform. Contacting is preferably performed in the presence of a mild base, preferably a non-nucleophilic one such as pyridine, lutidine, imidazole, or other nitrogenous bases with a pKa of about 4-7, more preferably of about 5-7. Contacting is preferably performed using an excess of phosphorous oxoacid derivative, preferably using about 2 to about 5 equiv. such as about 3 equiv. of reactant. After the reaction has proceeded, the reaction is preferably quenched using for example a basic aqueous solution, such as an aqueous (tri)alkylamine solution such as 1 volume of water and 1 volume of trimethylamine, which is then preferably left for about 10 to 60 minutes such as for about 30 minutes. The organic phase is preferably washed afterwards, such as with water. The organic phase is then preferably dried, such as by evaporation of solvent, preferably followed by co-evaporation, such as co-evaporation with toluene, ethyl acetate, or isopropanol, preferably with ethyl acetate and/or isopropanol.

The product of this contacting is a guanosine 5′-monophosphorous oxoacid ester analogue. This compound is preferably of general formula (II) or a salt thereof:

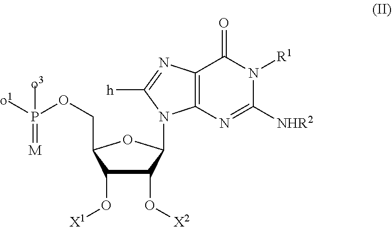

wherein h, X¹, X², R¹, and R² are as defined above; h is preferably Br; X1 is preferably H; X2 is preferably p′, more preferably (tri)alkylsilyl such as TIPS; R¹ is preferably H or forms —CH═C(ar)- together with R²; R² preferably forms —CH═C(ar)- together with R² or is H; most preferably R¹ and R² form —CH═C(ar)-
wherein o¹ and o³ are each independently —OH or as defined above; and
wherein M is S or O, preferably O. For compounds of general formula (II), definitions and preferred embodiments for h are preferably as defined for compounds of general formula (I). For compounds of general formula (II), definitions and preferred embodiments for Rd are preferably as defined for compounds of general formula (I). For compounds of general formula (II), definitions and preferred embodiments for R² are preferably as defined for compounds of general formula (I). For compounds of general formula (II), definitions and preferred embodiments for X¹ are preferably as defined for compounds of general formula (I). For compounds of general formula (II), definitions and preferred embodiments for X² are preferably as defined for compounds of general formula (I).

In preferred embodiments, o³ is H and o¹ is —OH, —O—C₁₋₈hydrocarbon, —S—C₁₋₈hydrocarbon, —NH—C₁₋₈hydrocarbon, borano, methylborano, dimethylborano, cyanoborano, or —N(C₁₋₈hydrocarbon)₂, more preferably —O—C₁₋₈hydrocarbon, —S—C₁₋₈hydrocarbon, —NH—C₁₋₈hydrocarbon, or —N(C₁₋₈hydrocarbon)₂. In other preferred embodiments, o and o³ are both —OH, —O—C₁₋₈hydrocarbon, —S—C₁₋₈hydrocarbon, —NH—C₁₋₈hydrocarbon, borano, methylborano, dimethylborano, cyanoborano, or —N(C₁₋₈hydrocarbon)₂, more preferably —O—C₁₋₈hydrocarbon, —S—C₁₋₈hydrocarbon, —NH—C₁₋₈hydrocarbon, or —N(C₁₋₈hydrocarbon)₂. In preferred embodiments, o¹ and o³ together form a chiral auxiliary as defined above. Most preferably, o¹ is —OH and o³ is H.

The following are preferred compounds of general formula (II):
1. 8-Bromoguanosine-5′-monophosphate,
2. 8-(2, 4-dihydroxyphenylthio)guanosine-5′-monophosphate,
3. 8-(2-aminophenylthio)guanosine-5′-monophosphate,
4. 8-(4-hydroxyphenylthio)guanosine-5′-monophosphate,
5. 8-(4-aminophenylthio)guanosine-5′-monophosphate,
6. 8-(4-chlorophenylthio)-3-phenyl-1,N²-ethenoguanosine-5′-monophosphate,
7. 8-(4-chlorophenylthio)guanosine-5′-monophosphate,
8. 8-(2, 4-dichlorophenylthio)guanosine-5′-monophosphate,
9. 8-(4-methoxyphenylthio)guanosine-5′-monophosphate,
10. 8-bromo-p-phenyl-1, N²-ethenoguanosine-5′-monophosphate,
11. 8-bromo-(2-naphthyl-1, N²-etheno)guanosine-5′-monophosphate,
12. 8-(4-hydroxyphenylthio)-3-phenyl-1,N²-ethenoguanosine-5′-monophosphate,
13. 8-(4-chlorophenylthio)-p-phenyl-1,N²-ethenoguanosine-5′-monophosphate,
14. 2-naphthyl- 1, N²-ethenoguanosine-5′-monophosphate,
15. β-phenyl-1, N²-ethenoguanosine-5′-monophosphate,
16. 4-methoxy-f3-phenyl-1,N²-ethenoguanosine-5′-monophosphate,
17. 8-Bromoguanosine-5′-mono-H-phosphonate,
18. 8-(2, 4-dihydroxyphenylthio)guanosine-5′-mono-H-phosphonate,
19. 8-(2-aminophenylthio)guanosine-5′-mono-H-phosphonate,
20. 8-(4-hydroxyphenylthio)guanosine-5′-mono-H-phosphonate,
21. 8-(4-aminophenylthio)guanosine-5′-mono-H-phosphonate,
22. 8-(4-chlorophenylthio)-3-phenyl-1,N²-ethenoguanosine-5′-mono-H-phosphonate,
23. 8-(4-chlorophenylthio)guanosine-5′-mono-H-phosphonate,
24. 8-(2, 4-dichlorophenylthio)guanosine-5′-mono-H-phosphonate,
25. 8-(4-methoxyphenylthio)guanosine-5′-mono-H-phosphonate,
26. 8-bromo-p-phenyl-1, N²-ethenoguanosine-5′-mono-H-phosphonate,
27. 8-bromo-(2-naphthyl-1, N²-etheno)guanosine-5′-mono-H-phosphonate,
28. 8-(4-hydroxyphenylthio)-3-phenyl-1,N²-ethenoguanosine-5′-mono-H-phosphonate,
29. 8-(4-chlorophenylthio)-p-phenyl-1,N²-ethenoguanosine-5′-mono-H-phosphonate,
30. 2-naphthyl- 1, N²-ethenoguanosine-5′-mono-H-phosphonate,
31. β-phenyl-1, N²-ethenoguanosine-5′-mono-H-phosphonate,
32. 4-methoxy-f3-phenyl-1,N²-ethenoguanosine-5′-mono-H-phosphonate,
33. β-1, N²-acetyl-8-bromoguanosine-5′-monophosphate and its mono-H-phosphonate,
34. 8-Bromo-δ-1,N²-butyrylguanosine-5′-monophosphate and its mono-H-phosphonate,
35. 8-bromo-(4-methyl-β-phenyl-1,N²-etheno)guanosine-5′-monophosphate and its mono-H-phosphonate,
36. 8-bromo-(3-thiophen- yl- 1, N²-etheno)guanosine-5′-monophosphate and its mono-H-phosphonate
37. 1-Benzyl- 8-bromoguanosine-5′-monophosphate and its mono-H-phosphonate
38. 8-Thioguanosine-5′-monophosphate and its mono-H-phosphonate,
39. 8-(4-Isopropylphenylthio)guanosine-5′-monophosphate and its mono-H-phosphonate,
40. 8-Phenylamidomethylthioguanosine-5′-monophosphate and its mono-H-phosphonate,
41. β-phenyl- 1, N²-etheno- 8-phenylamidomethylthioguanosine-5′-monophosphate and its mono-H-phosphonate,
42. 8-(4-Isopropylphenylthio)-p-phenyl-1, N²-ethenoguanosine-5′-monophosphate and its mono-H-phosphonate,
43. 8-(2-Aminophenylthio)- β-phenyl- 1, N²-ethenoguanosine-5′-monophosphate and its mono-H-phosphonate,
44. 3-Phenyl- 1, N²-etheno-8-thioguanosine-5′-monophosphate and its mono-H-phosphonate,
45. 8-Methylthio- β-phenyl- 1, N²-ethenoguanosine-5′-monophosphate and its mono-H-phosphonate,
46. 8-methylthio-guanosine-5′-monophosphate, preferably a sodium salt, and its mono-H-phosphonate,
47. 8-Phenylguanosine-5′-monophosphate and its mono-H-phosphonate,
48. 8-(2-Furyl)guanosine-5′-monophosphate and its mono-H-phosphonate,
49. 8-(4-Chlorophenyl)guanosine-5′-monophosphate and its mono-H-phosphonate,
50. 8-Phenyl-β-phenyl- 1, N²-ethenoguanosine-5′-monophosphate and its mono-H-phosphonate,
51. 8-(4-Chlorophenyl)- β-phenyl- 1, N²-ethenoguanosine-5′-monophosphate and its mono-H-phosphonate,
   or salts thereof.

Reference to a compound from the above list is intended to also refer to a salt of such a compound, wherein salts are as defined elsewhere herein. In preferred embodiments the free base compound is referenced. In preferred embodiments a salt is referenced. In more preferred embodiments the compound is selected from compounds 1-16. In other more preferred embodiments the compound is selected from compounds 17-32. In other preferred embodiments the compounds are selected from 1, 10, 11, 17, 26, and 27, more preferably from 1, 10, and 11, alternatively more preferably from 17, 26, and 27. In other preferred embodiments the compounds are selected from 6, 10-16, 22, and 26-32, more preferably from 6 and 10-16, alternatively more preferably from 22 and 26-32. In other preferred embodiments the compounds are selected from 6, 10-13, 15, 16, 22, 26-29, 31, and 32, more preferably from 6, 10-13, 15, and 16, alternatively more preferably from 22 and 26-29, 31, and 32. In other preferred embodiments the compounds are selected from 1-5, 7-9, 17-21, and 23-25, more preferably from 1-5 and 7-9, alternatively more preferably from 17-21 and 23-25. Very highly preferred compounds are compound 10 and 26, most preferably compound 26. Other very highly preferred compounds are compounds 10, 35, 36, and 40-43 and their mono-H-phosphonates, more preferably their mono-H-phosphonates.

Step iii) Crystallizing the Guanosine 5′-Monophosphorous Oxoacid Ester Analogue

The inventors surprisingly found that the 5′-monophosphorous oxoacid ester analogue produced in step ii) can be crystallized. This intermediate is never isolated in cGMP production methods known in the art, while purification of this intermediate was found to have advantageous effects, such as increase in cyclisation yield and improved control over the chirality of the cyclisation product. Preferably, the guanosine 5′-monophosphorous oxoacid ester analogue is a salt when it is crystallized, more preferably it is a salt wherein the counterion is a hydronated organic base, most preferably wherein the counterion is a hydronated (tri)alkylamine such as TEAH+.

The guanosine 5′-monophosphorous oxoacid ester analogue is isolated by crystallization, preferably from an aprotic organic solvent, more preferably from a polar one, such as ethyl acetate or propyl acetate or THE or tert-butyl methyl ether, most preferably from ethyl acetate. The crystallization is preferably seeded, more preferably using the desired product to be crystallized. After crystallization, which is preferably left for at least 4, more preferably at least 8, even more preferably at least 12 hours, the crystals are preferably washed and/or dried. Crystallization is preferably at room temperature. Washing is preferably performed using the same solvent as was used for the crystallization. Drying is preferably performed in vacuum, more preferably at elevated temperature such as at about 40-80° C., preferably at about 50-70° C. In preferred embodiments the obtained crystal is a solvate. Preferred crystals comprise substantially pure product, more preferably having a purity of at least 80, 85, 90, 91, 92, 93, 94, 95, 96, 97, 98, 99, or 100%, even more preferably of at least 95, 96, 97, 98, 99, or 100%, most preferably of at least 97%, optionally more.

### Step iv) Formation of the Cyclic Diester

The 5′-phosphate monoester isolated in step iii) can advantageously be used to continue synthesis of a cGMP analogue. Accordingly, preferred methods according to the invention further comprise a step:
iv) cyclizing the guanosine 5′-monophosphorous oxoacid ester analogue obtained in step ii) (and crystallized in step iii) to obtain a cyclic guanosine-3′, 5′-monophosphate (cGMP) analogue, wherein said cyclisation is preferably performed in the presence of a sterically hindered base. It stands to reason that if X¹ is not H, this step is preferably preceded by conversion of X¹ to H by deprotecting the corresponding protected hydroxyl moiety.

A cGMP analogue is preferably is of general formula (III) or a salt thereof:

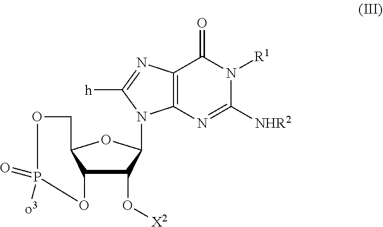

wherein h, X², R¹, and R² are as defined above, preferably as defined for compounds of general formula (II). Preferably X² is p′, more preferably it is (tri)alkylsilyl such as TIPS. o³ is as defined for compounds of general formula (II), preferably o³ is H. In other preferred embodiments, o³ is —OH, —O—C₁₋₈hydrocarbon, —S—C₁₋₈hydrocarbon, —NH—C₁₋₈hydrocarbon, borano, methylborano, dimethylborano, cyanoborano, or —N(C₁₋₈hydrocarbon)₂, most preferably —OH.

For compounds of general formula (III), definitions and preferred embodiments for h are preferably as defined for compounds of general formula (II). For compounds of general formula (III), definitions and preferred embodiments for R¹ are preferably as defined for compounds of general formula (II). For compounds of general formula (III), definitions and preferred embodiments for R² are preferably as defined for compounds of general formula I(I). For compounds of general formula (III), definitions and preferred embodiments for X² are preferably as defined for compounds of general formula (II).

Cyclisation can be performed using methods known in the art. Preferably, cyclisation is performed in the presence of a coupling reagent. Preferably, cyclisation is performed in the presence of a sterically hindered base or pyridine, more preferably of a sterically hindered mild base or pyridine, even more preferably of a sterically hindered mild base. Most preferably, both a coupling reagent and a sterically hindered base are used. The cyclisation is preferably performed under dilute conditions, such as at most about 200 g/L, preferably at most about 100 g/L, more preferably at most about 80 g/L, even more preferably at most about 60 g/L such as about 50 g/L, expressed as weight of 5′-monophosphorous oxoacid ester analogue per liter of solvent. The solvent is preferably an aprotic solvent, more preferably a slightly polar one such as dichloromethane or chloroform or acetonitrile, even more preferably chloroform or dichloromethane. As a skilled person is aware, under dilute conditions, the base is preferably present in excess, such as at about 2-9 equiv., more preferably about 4-6 equiv., such as about 5 equiv. Similarly, the coupling reagent is preferably present in excess, such as at about 1.1-5 equiv., more preferably about 1.2-3 equiv., such as about 1.5 equiv. Preferably, the base is added to the dilute 5′-monophosphorous oxoacid ester analogue solution first, after which the coupling reagent is added. The reaction is preferably allowed to proceed for at least 30 minutes, more preferably at least 1 hour, most preferably about 2 hours or more.

Preferred examples of suitable sterically hindered bases are lutidine, picoline, collidine, N,N-dimethylaniline, N-methyl-morpholine, and quinolone, more preferably lutidine. Further preferred sterically hindered bases have a pKa of about 4-7, more preferably of about 5-7.

Suitable coupling reagents are widely known in the art. Preferred examples of suitable coupling reagents are aryl sulfonic acid derivatives, diester chlorophosphates, carbodiimides, and acyl halides, such as O-(benzotriazol-1-yl)-N,N,N′,N′-tetramethyluronium hexafluorophosphate (HBTU), bis(2-oxo-3-oxazolidinyl)phosphinic chloride (BOP-Cl), 2-chloro-4,6-dimethoxy-1,3,5-triazine (CDMT), (chloromethylene)dimethyliminium chloride (Vilsmeier reagent), N-(dimethylaminopropyl)-N′-ethyl-carbodiimide (EDC), carbonyl diimidazole (CDI), propylphosphonic anhydride (T3P), diethyl chlorophosphate, dicyclohexylcarbodiimide (DCC), isobutyl chloroformate, and pivaloyl chloride. More preferably acyl halides are used, of which acyl chlorides are most preferred, such as propyl chloride, benzoyl chloride, ethyl chloroformate, isobutyl chloroformate, and pivaloyl chloride. Pivaloyl chloride is most highly preferred.

Preferably, the cGMP analogue can be isolated by washing the organic phase, preferably with water, after which it is preferably diluted with an aprotic polar solvent such as acetonitrile. Preferably, the organic phase is evaporated. The obtained crude product is preferably further purified by resuspending the crude solids in an aprotic polar solvent such as acetonitrile, after which solids are filtered out. The filtrate is then preferably concentrated in vacuo, after which it is preferably redissolved in an aprotic polar solvent such as acetonitrile. A crude solution is then preferably treated by addition of an excess of an ether solvent, preferably a tert-butyl ether solvent such as tert-butyl methyl ether (TBME). The excess is preferably at least 5 volumes, more preferably at least 10 volumes such as about 12 volumes. The resulting composition is preferably left for at least about 4 hours, more preferably at least about 8 hours, after which solvent is removed, preferably by decantation and/or evaporation. The resulting residue can be an oil. The resulting residue is preferably dried in vacuo, after which it is preferably triturated using an ether solvent, preferably a tert-butyl ether solvent such as TBME. In other highly preferred embodiments, preferably when cyclisation is to be followed by phosphorothioate formation, the cGMP analogue is not isolated prior to any further reaction steps.

The inventors surprisingly found that use of a sterically hindered base led to very desirable stereochemical outcome of the reaction, that is it led to a large diastereomeric excess of the Rp-stereoisomer, which is the preferred stereoisomer. Rp- and Sp-stereoisomers of general formula (III) are shown below, and are of general formula (III-Rp) and (III-Sp) respectively. Without wishing to be bound to theory, it is possible that use of a sterically hindered base leads to an overall slower reaction, or a reaction under kinetic control, which results in a preferred formation of kinetic product, which is the Rp-product.

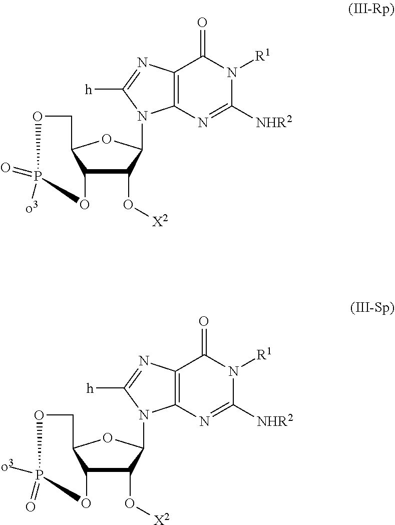

In preferred embodiments, a compound of general formula (III) is of general formula (III-Rp). Because for different applications the different diastereomers can be relevant, in other preferred embodiments a compound of general formula (III) is of general formula (III-Sp). In compounds of general formula (III-Rp) or (III-Sp), h, X², R¹, and R² are preferably as defined above, more preferably as defined for compounds of general formula (III). Preferably X² is p′, more preferably (tri)alkylsilyl, most preferably TIPS. o³ is preferably as defined for compounds of general formula (II), more preferably it is as defined there but not —OH, even more preferably o³ is H or SH. As an intermediary product produced in step iv, most preferably o³ is H.

Preferably, when a compound is of general formula (III-Rp) or of general formula (III-Sp), the corresponding diastereomer is present in a diastereomeric excess of at least 2:1, more preferably of at least 3:1, even more preferably at least 4:1, more preferably still at least 5:1, even more preferably still at least 6:1, still more preferably at least 7:1, most preferably at least about 8:1. Preferably, when a compound of general formula (III) and/or of general formula (III-Rp) or (III-Sp) is obtained after step iv), the compound has a purity of at least 60, 65, 70, 75, 80, 85, 90, 95, 96, 97, 98, 99, or more percent. More preferably purity is at least 70%, even more preferably at least 75%, more preferably still at least 80%, even more preferably still at least 85%, still more preferably at least 90%. Purity can be assayed using known techniques, preferably it is assayed using HPLC.

Preferred compounds of general formula (III) are a salt thereof, more preferably a salt wherein the counterion is a hydronated organic base, most preferably wherein the counterion is a hydronated (tri)alkylamine such as TEAH+. Preferred compounds of general formula (III-Rp) are a salt thereof, more preferably a salt wherein the counterion is a hydronated organic base, most preferably wherein the counterion is a hydronated (tri)alkylamine such as TEAH+. Preferred compounds of general formula (III-Sp) are a salt thereof, more preferably a salt wherein the counterion is a hydronated organic base, most preferably wherein the counterion is a hydronated (tri)alkylamine such as TEAH+.

### Step v) Phosphorothioate Formation

Phosphorothioates are useful cGMP analogues. Accordingly, in preferred embodiments, the method according to the invention comprises a step v):

v) contacting the cGMP analogue with a sulfurizing agent to obtain a thiolated cGMP analogue of general formula (III) wherein o³ is —SH or —S—C₁₋₁₂hydrocarbon, preferably —SH. In preferred embodiments the cGMP analogue is of general formula (III-Rp). In other preferred embodiments, the cGMP analogue is of general formula (III-Sp). Step v) is preferred when M is O in a guanosine 5′-monophosphorous oxoacid analogue of general formula (II). When for such a precursor of general formula (II) M is S, preferably step v) is not comprised in the method.

Sulfurizing agents are known in the art. Preferred examples of sulfurizing agents are sulfur, phenylacetyldisulfide and N-(alkyl-thio)-succinimides such as N-methylthio-succinimide. N-ethylthio-succinimide, and N-propylthio-succinimide. Sulfur is most preferred. Preferred C₁₋₁₂hydrocarbons are C₁₋₈hydrocarbons, more preferably C₁₋₄hydrocarbons. Examples of preferred —S—C₁₋₁₂hydrocarbons are —S—CH₃, —S—CH₂CH₃, —S—CH(CH₃)₂, —S—C(CH₃)₃, and —S-phenyl; —S—CH₃ is most preferred. Suitable sulfur introduction comprises using electrophilic sulfur, preferably the use of a sulfurizing agent as described above, followed by oxidation (i.e. an increase in oxidation state), preferably using an oxidizing agent as described below. Use of nucleophilic sulfur is not preferred.

Preferably, the contacting is performed in the presence of a base, more preferably a (tri)alkylamine base such as triethylamine. The reaction is preferably allowed to proceed for at least 30 minutes, more preferably at least 60 minutes, most preferably at least 90 minutes. Progress of the reaction is preferably assessed using known techniques such as HPLC.

Alternately, when this step is performed starting from cGMP analogues wherein o³ is H, it can be useful to convert the P(III) to P(V). Accordingly, in preferred embodiments, the method according to the invention comprises a step v-O):

v-O) contacting the cGMP analogue with an oxidizing agent to obtain a cGMP analogue of general formula (III) wherein o³ is —OH. Such oxo-derivatives are activators of PKG and not inhibitors thereof, such as the thiophosphates wherein o³ comprises S. Features and conditions are preferably as described for step v). Oxidizing agents are known in the art. Step v-O) is preferably performed under anhydrous conditions. Examples of suitable oxidizing agents for step v-O) are peroxides such as hydrogen peroxide, bis(trimethylsilyl)peroxide, t-butyl hydroperoxide, cumene hydroperoxide, iodine, and alkylamino-oxides such as N-methylmorpholine N-oxide. Alternately, the agent can be catalytic, such as Pd/C powder. Examples of phosphite oxidation to phosphates are known, for example from Hayakawa et al. (Tet. Lett. 1986, DOI: 10.1016/S0040-4039(00)₈₄₉₄₆-1) using peroxides, and from Nagaosa and Aoyama (Carbon, 2001, DOI: 10.1016/S0008-6223(01)00206-8) using Pd/C.

Preferably, when a compound of general formula (III) and/or of general formula (III-Rp) or (III-Sp) is obtained after step v) or v-O), the compound has a purity of at least 60, 65, 70, 75, 80, 85, 90, 95, 96, 97, 98, 99, or more percent. More preferably purity is at least 70%, even more preferably at least 75%, more preferably still at least 80%, even more preferably still at least 85%, still more preferably at least 90%. Purity can be assayed using known techniques, preferably it is assayed using HPLC.

Step vi) cGMP Analogue Deprotection

To obtain products with interesting pharmacological properties, the cGMP analogues produced by the method of the invention are preferably deprotected, which in the context of this invention means that produced cGMP analogues of general formula (III) preferably have X² is H. Therefore, in preferred embodiments is provided the method according to the invention wherein X² is p′, the method further comprising the step of:

vi) deprotecting the hydroxyl moiety that is protected by X² to obtain a deprotected cGMP analogue. A deprotected cGMP analogue is a cGMP analogue of general formula (III) wherein X² is H. Means of deprotection are well-known in the art, and depend on the nature of p′. Generally, deprotection can be performed at acidic conditions, for example at pH<1, optionally at elevated temperatures. Deprotection can also preferably be performed in the presence of fluoride, for example using HF, (tri)alkylamine-HF, or TBAF. For deprotection of (tri)alkylsilyl protecting groups, deprotection in the presence of fluoride is preferred, more preferably using (tri)alkylamine-HF such as triethylamine HF, preferably at 10-50 vol.-% of the solution, more preferably at 20-40 vol.-% such as at about 33 vol.-%. Deprotection is preferably performed in an aprotic solvent, more preferably a polar aprotic solvent that is a cyclic ether, such as tetrahydrofuran or 1,4-dioxane. The reaction is preferably allowed to proceed for about a day, more preferably about 2 days, even more preferably about 3 days. Standard analytical techniques such as HPLC can be used to monitor reaction progress. Precipitated deprotected product is preferably isolated by filtration, after which it is preferably washed, more preferably using the solvent that was used for deprotection. Afterwards the product is preferably dried, such as under vacuum. Products obtained this way are a fluoride complex of the deprotected cGMP analogue. In preferred embodiments, a produced cGMP analogue is in a complex with fluoride. This cGMP analogue fluoride complex preferably has a purity, as assayed for the cGMP analogue, of at least 60, 65, 70, 75, or 80 percent. More preferably purity is at least 70%, even more preferably at least 75%, more preferably still at least 80%. Purity can be assayed using known techniques, preferably it is assayed using HPLC. This compound is preferably a salt, more preferably a (tri)alkylamine salt, most preferably a TEAH+ salt.

Step vii) Triturating the Deprotected cGMP Analogue

In preferred embodiments, the method according to the invention comprises the step viii):
vii) triturating the deprotected cGMP analogue.

A deprotected cGMP analogue is as described above. This trituration is preferably to remove fluoride from a cGMP analogue fluoride complex. However, the inventors also surprisingly found that the trituration revealed an improved diastereomeric excess of isolated compounds of general formula (III-Rp), and accordingly the invention provides a method for the production of a cGMP analogue, comprising step vi), more preferably comprising steps vi) and vii), wherein features and definitions are as defined elsewhere herein; preferably this method also comprises step viii) as described below.

Preferably, for trituration the deprotected cGMP analogue can be resuspended in a polar solvent, preferably in an aprotic polar solvent such as acetonitrile. It is then preferably filtered and washed, more preferably washed using the solvent in which it was resuspended. The resulting cGMP analogue preferably has a purity of at least 60, 65, 70, 75, 80, 85, 90, 95, 96, 97, 98, 99, or more percent. More preferably purity is at least 75%, even more preferably at least 80%, more preferably still at least 85%, even more preferably still at least 90%, still more preferably at least about 95%. This deprotected cGMP analogue is preferably substantially free of fluoride, more preferably entirely free of fluoride. Absence of fluoride can be confirmed using ¹⁹F NMR. Purity can be assayed using known techniques, preferably it is assayed using HPLC. This compound is preferably a salt, more preferably a (tri)alkylamine salt, most preferably a TEAH+ salt.

Step viii) Formation of a Pharmaceutically Acceptable Salt

For pharmaceutical applications it is preferred that the cGMP analogue is either a free base or a pharmaceutically acceptable salt, more preferably a pharmaceutically acceptable salt. Accordingly, preferred methods according to the invention comprise a step viii):
viii) converting the deprotected cGMP analogue to a pharmaceutically acceptable salt, preferably a sodium salt. Features and definitions have been provided above. A cGMP analogue converted in step viii) is preferably a product of step vi) or of step vii), most preferably of step vi) followed by step vii). This compound is most preferably a salt, more preferably a (tri)alkylamine salt, most preferably a TEAH+ salt.

Preferably the cGMP analogue is admixed with a protic solvent, more preferably a lower alcohol such as methanol. This preferably results in a suspension. This is preferably followed by addition of an alkoxide salt of which the counterion is the intended counterion for the pharmaceutically acceptable cGMP salt, preferably an alkoxide salt of the solvent and an alkali metal such as potassium or sodium, more preferably sodium. A preferred alkoxide salt is sodium methoxide. The resulting solution is preferably concentrated in vacuo to obtain a pharmaceutically acceptable salt of the cGMP analogue, preferably the sodium salt.

After this concentration the resulting solids are preferably stirred with a lower alcohol, more preferably with ethanol, after which solids are separated by filtration. The inventors surprisingly found that after filtration, the mother liquor contained a smaller fraction of Sp-diastereomer than the solids that were filtered out), and accordingly the invention provides a method for the production of a cGMP analogue, comprising step viii) wherein features and definitions are as defined elsewhere herein; preferably this method also comprises step vii) as described above.

In preferred embodiments, the solids are stirred with a slightly wet lower alcohol, more preferably with a slightly wet ethanol, most preferably with 96% ethanol (wherein the remaining 4%, both being vol.-%, is preferably substantially water), for at least 1, more preferably at least 2 hours. After this, the filtrate is preferably concentrated in vacuo. Purity of the resulting solids is preferably further increased by further suspension in dry lower alcohol, preferably in dry ethanol, after which the suspension is filtered and the solids are preferably washed with additional dry alcohol, preferably the same alcohol used for resuspension. Afterwards the obtained solids are preferably dried, such as vacuum dried.

Preferably, the pharmaceutically acceptable salt of a cGMP analogue comprises at most 10% of the undesired diastereomer, more preferably at most 5%, even more preferably at most 4, 3, or 2%, even more preferably at most 1% such as at most 0.5%. Preferably the pharmaceutically acceptable salt of a cGMP analogue has a purity of at least 80, 85, 90, 91, 92, 93, 94, 95, 96, 97, 98, 99, or more percent. More preferably purity is at least 90%, even more preferably at least 94%, more preferably still at least 95%, even more preferably still at least 96%, still more preferably at least about 97%. Purity can be assayed using known techniques, preferably using NMR.

In highly preferred embodiments is provided the method according to the invention wherein X² is p′, the method further comprising the steps of:
vi) deprotecting the hydroxyl moiety that is protected by X2 to obtain a deprotected cGMP analogue; and
vii) optionally triturating the deprotected cGMP analogue; and
viii) optionally converting the deprotected cGMP analogue to a pharmaceutically acceptable salt, preferably a sodium salt.

### Products and Intermediates Produced by the Method

The method according to the invention, besides offering an advantageous path towards cGMP analogues, also allows efficient formation of several key intermediates. In another aspect the invention provides several of these compounds, preferably a compound of general formula (II) or a salt thereof, wherein h, X¹, X², R¹, and R² are as defined above; wherein o¹ and o³ are each independently —OH or as defined for compounds of general formula (P), wherein preferably o³ is H or o¹ and o³ together form a chiral auxiliary that is preferably a C₂₋₁₂hydrocarbon; wherein M is S or O; wherein preferably the compound is a salt. The products can be used in the manufacture of a medicament, preferably a medicament for the treatment of retinitis pigmentosa.

Further features and definitions are as described in the first aspect above for compounds of general formula (II). Accordingly, in preferred embodiments within this aspect, o³ is H and o¹ is —OH, —O—C₁₋₈hydrocarbon, —S—C₁₋₈hydrocarbon, —NH—C₁₋₈hydrocarbon, borano, methylborano, dimethylborano, cyanoborano, or —N(C₁₋₈hydrocarbon)₂, more preferably —O—C₁₋₈hydrocarbon, —S—C₁₋₈hydrocarbon, —NH—C₁₋₈hydrocarbon, or —N(C₁₋₈hydrocarbon)₂. In other preferred embodiments, o¹ and o³ are both —OH, —O—C₁₋₈hydrocarbon, —S—C₁₋₈hydrocarbon, —NH—C₁₋₈hydrocarbon, borano, methylborano, dimethylborano, cyanoborano, or —N(C₁₋₈hydrocarbon)₂, more preferably —O—C₁₋₈hydrocarbon, —S—C₁₋₈hydrocarbon, —NH—C₁₋₈hydrocarbon, or —N(C₁₋₈hydrocarbon)₂. In preferred embodiments, o¹ and o³ together form a chiral auxiliary as defined above. Most preferably, o¹ is —OH and o³ is H.

Preferably, the compounds within this aspect are crystalline. The intermediate can also be a hydrate or a solvate. In preferred embodiments the crystal is a solvate, more preferably an isopropyl acetate solvate. In other embodiments, the crystal is a hydrate. In highly preferred embodiments the crystal is not a solvate or a hydrate. In preferred embodiments the crystal comprises fluoride. In preferred embodiments, the crystal comprises substantially pure intermediate, more preferably having a purity of at least 80, 85, 90, 91, 92, 93, 94, 95, 96, 97, 98, 99, or 100%, even more preferably of at least 95, 96, 97, 98, 99, or 100%, most preferably of at least 97% or optionally more.

In preferred embodiments the invention provides a compound of general formula (II) or a salt thereof, wherein h, X¹, X², R¹, and R² are as defined above; wherein o¹ is —OH or as defined for compounds of general formula (P), wherein o³ is H; wherein M is S or O; wherein preferably the compound is a salt, more preferably a TEAH+ salt. Further features and definitions are as described in the first aspect above for compounds of general formula (II).

In preferred embodiments the invention provides a compound of general formula (II) or a salt thereof, wherein h, X¹, X², R¹, and R² are as defined above; wherein or o¹ and o³ together form a chiral auxiliary that is preferably a C₂₋₁₂hydrocarbon; wherein M is S or O; wherein preferably the compound is a salt. Further features and definitions are as described in the first aspect above for compounds of general formula (II).

In preferred embodiments the invention provides a compound of general formula (II) or a salt thereof, wherein h is Br; X¹ is H and X² is p′; p′ is preferably a (tri)alkylsilyl, more preferably triisopropylsilyl (TIPS); R¹ and R² together form —CH═C(ar)- wherein ar is preferably most proximal to R²; ar is phenyl or naphthyl, preferably phenyl; o¹ is OH; o³ is H; and M is S or O, preferably O.

A desirable product of the method is of general formula (III), more preferably of general formula (III-Rp), wherein o³ is —SH (or optionally is —S when the compound is a sodium salt), wherein X² is H, wherein h is —Br, and wherein R¹ and R² together form —CH═C(ar)- wherein ar is preferably most proximal to R², and ar is 4-methylphenyl.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value more or less 1% of the value.

An oxoacid is also known as an oxyacid and is a compound that contains hydrogen, oxygen, and at least one atom of a further element, with at least one hydrogen atom bonded to oxygen that can dissociate to produce the H⁺ cation and the anion of the acid. A phosphorous oxoacid is an oxoacid wherein the further element is phosphorous.

Molecules provided in this invention can be optionally substituted. Suitable optional substitutions are replacement of —H by a halogen. Preferred halogens are F, Cl, Br, and I. Further suitable optional substitutions are substitution of one or more —H by —NH₂, —OH, ═O, alkyl, alkoxy, haloalkyl, haloalkoxy, alkene, haloalkene, alkyn, haloalkyn, and cycloalkyl. Alkyl groups have the general formula CₙH₂ₙ₊₁ and may alternately be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of alkyl groups include methyl, ethyl, propyl, 2-propyl, t-butyl, 1-hexyl, 1-dodecyl, etc.

Unless stated otherwise, —H may optionally be substituted with one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups, wherein the silyl groups can be represented by the formula (R^{s})₃Si—, wherein R^{s} is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

When a structural formula or chemical name is understood by the skilled person to have chiral centers, yet no chirality is indicated, for each chiral center individual reference is made to all three of either the racemic mixture, the pure R enantiomer, and the pure S enantiomer. When two moieties are said to together form a bond, this implies the absence of these moieties as atoms, and compliance of valence being fulfilled by a replacing electron bond. Dashes in representations of moieties mainly serve to guide the reader; as a non-limiting example —NH—(CH₂)ₙ—OH and —NH—(CH₂)ₙOH refer to the same moiety. All this is known in the art.

Whenever a parameter of a substance is discussed in the context of this invention, it is assumed that unless otherwise specified, the parameter is determined, measured, or manifested under physiological conditions. Physiological conditions are known to a person skilled in the art, and generally comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use are suitable for use in methods of treatment.

Filtration can be performed using any method known in the art, such as preferably using a Buchner funnel or an agitated Nutsche filter.

The present invention has been described above with reference to a number of exemplary embodiments. Modifications and alternative implementations of some parts or elements are possible, and are included in the scope of protection as defined in the appended claims. All citations of literature and patent documents are hereby incorporated by reference.

### DESCRIPTION OF DRAWINGS

FIG. 1—synthetic route towards Rp-8-Br-PET-cGMPS.

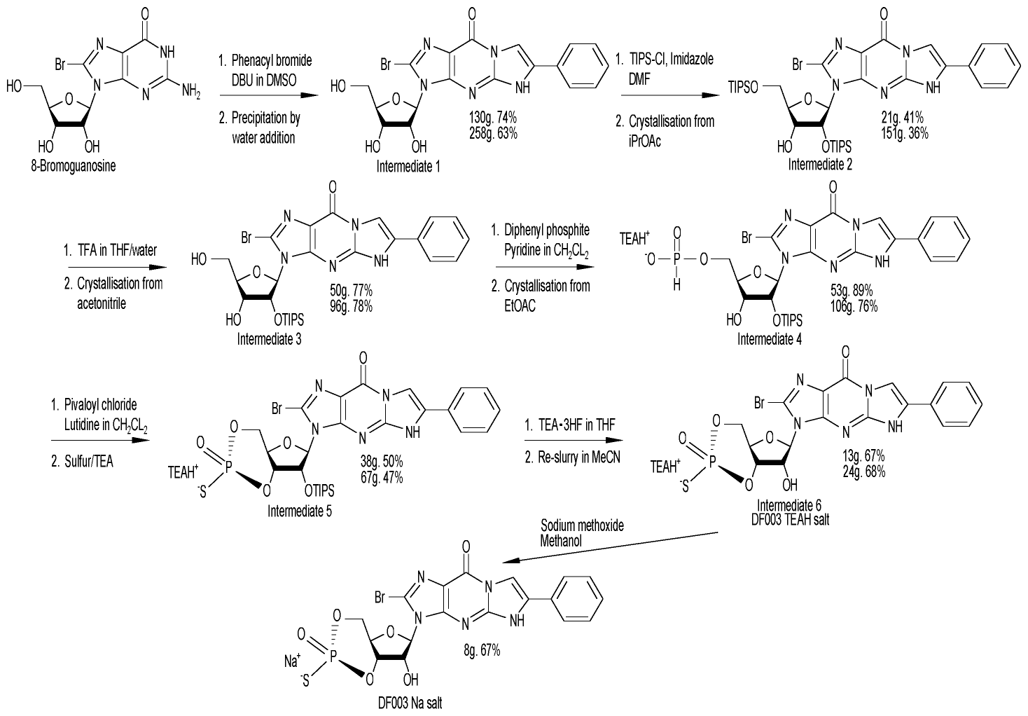

### EXAMPLES

### Example 1—Synthesis of Rp-8-Br-PET-cGMPS

### 1.1 PET Formation

152 g 8-Bromoguanosine (90% assay, remainder water) was stirred in 5 vol DMSO (760 ml) and 1.5 equiv. phenacyl bromide was added to the suspension. Next, 2.5 equiv. of DBU was added during 30 min. After 1 hour 3 equiv (65 ml) of acetic acid was added. The product was precipitated by slow addition of 10 vol water (1520 ml). The slurry was filtered and washed with 2000 ml DMSO:water 1:2 followed by 200 ml water. The cake was vacuum dried, re-suspended in 2500 ml MeCN and stirred overnight. After filtration and wash with MeCN, vacuum drying at 70° C. gave 130 g (74%) of Intermediate 1.

### 1.2 Protection of the Guanosine Analogue

130 g Br-PET-Guanosine (Intermediate 1) was suspended in 5 vol DMF (650 ml). 5 equiv. imidazole (96 g) and 2.5 equiv. TIPS—Cl (151 ml) was added. The reaction was analyzed after 48h (HPLC indicated 1.08% 5′-monoTIPS, 67.76% 5′2′-diTIPS, 29.52% 5′3′-diTIPS, and 1.64% 5′2′3′-triTIPS). The reaction was quenched with 50 ml water (10 equiv.) and stirred for 1 h. After dilution with 15 vol toluene (1900 ml) the reaction was washed with 3*5 vol water (650 ml each). The organic phase was evaporated and the sticky residue was dissolved in 20 volumes i-propyl acetate (2300 ml), seeded and left to crystallize overnight. Filtration and wash with i-propyl acetate gave after vacuum drying 81 g (37%) of Intermediate 2 (98.5% purity by HPLC).

1.3 selective Deprotection

150 g of 5′-,2′-DiTIPS-Br-PET-Guanosine (Intermediate 2) was dissolved in a 10 vol THE (1500 ml). 2 vol water (300 ml) and 0.45 vol TFA (67.5 ml) was then added. After stirring overnight (reaction checked, HPLC indicated 3.88% double deprotection, 94.75% of the desired 2′-TIPS product, and 1.37% unconverted product) 0.4 vol 28% ammonia (aq.) (60 ml) was added (pH became 5) and the mixture was concentrated. 15 vol CH₂Cl₂ (2250 ml) and 2 vol water (300 ml) was added and the resulting thin slurry was filtered (this removes overhydrolyzed material, i.e. Br-PET-Guanosine). The organic phase was separated and evaporated. To the residue 3 vol acetonitrile (450 ml) was added and the slurry was stirred for 1 hour before filtration and wash with 2 vol acetonitrile (300 ml). Vacuum drying gave 96 g (78%) of Intermediate 3, which assayed at 100% purity by HPLC.

### 1.4 5′-Monoester Formation

111 g of 2′-TIPS-Br-PET-Guanosine (Intermediate 3) was dissolved in a mixture 19 vol CH₂Cl₂ (1945 ml) and 1 vol pyridine (110 ml). Next, with stirring 3 equiv. (102 ml) of diphenyl phosphite was added. After 2 hours 1 vol of water (110 ml) and 1 vol of triethylamine (110 ml) were added and the reaction was left for 30 min and then washed with 2*10 volumes (2*1100 ml) of water. The organic phase was evaporated and the residue was co-evaporated with 500 ml i-propanol followed by 500 ml ethyl acetate. The residual oil was then dissolved in 1500 ml ethyl acetate, seeded and left to crystallize overnight. Filtration and wash with ethyl acetate gave after vacuum drying 106 g (76%) of Intermediate 4. The product does not behave well on a reversed phase liquid chromatography system, giving a broad and late peak (H₂O:MeCN, 0.1% formic acid), but it assays at 97.56%.

1.5 Cyclisation to Form a cGMP Analogue

50.5 g of 5′-Phosphonate-2′-TIPS-BrPETGuanosine was dissolved in 1000 ml CH₂Cl₂ (20 vol.) followed by addition of 37 ml 2,6-lutidine (5 equiv.). Next, 11.8 ml pivaloyl chloride was added. The reaction was left to proceed for two hours.

### 1.6 Formation of a Phosphorothioate

To the crude reaction solution of example 1.5, 5.9 g of sulfur (3 equiv.) was added followed by 26 ml of triethylamine. After 1.5h (HPLC indicated 74.05% Rp-diastereomer and 9.72% Sp-diastereomer with the remainder being impurities) the solution was washed with 2*200 ml water, the organic phase was diluted with 200 ml MeCN and evaporated. The residue (100 g) was slurried in 400 ml MeCN for 10 min and filtered. The filtrate was evaporated and re-dissolved in 100 ml MeCN. Slow addition of 1200 ml TBME and stirring overnight gave some oil and sticky precipitation. After decantation and evaporation and the solution, the remaining oil was thoroughly dried by high vacuum forming a sticky foam. The foam was stirred and triturated with 700 ml TBME, yielding after filtration and drying 38 g yellow powder. The assay, calculated as the TEAH salt of the desired diastereomer, was 68% giving a corrected yield of 50%. HPLC purity was 80% (260 nm) and the diasteromeric ratio was 8:1.

1.7 Deprotection of the cGMP Analogue

35 g of Intermediate 5 (68% assay of Rp-diastereomer) was dissolved in 4 vol THE (140 ml) and 2 vol TEA*3HF (70 ml) was added. The reaction was stirred for 3 days (HPLC indicated 70.68% deprotection), filtered and washed with 3 vol THE (105 ml) and vacuum dried. 17.5 g of product with an assay of 83% (76% yield) was obtained. The material is some kind of fluoride containing complex (as seen by ¹⁹F NMR, and evident by the low assay). The material was slurried in 300 ml MeCN, filtered and washed with 150 ml MeCN (this MeCN reslurry removed the fluoride seen by NMR and improved overall purity, as HPLC indicated 97.13% of the desired Rp-product, and only 0.78% of the undesired Sp-product, whereas the mother liquor comprised 76.83% Rp-product and 14.53% of the undesired Sp-product). This gave 13.5 g of material with an assay of 95%, 67% yield). The material contained no fluoride according to ¹⁹F NMR.

### 1.8 Sodium Salt Formation

13 g Intermediate 6 (8-Br-PET-cGMP TEAH salt, 95% NMR assay) was suspended in 500 ml MeOH. 1 equiv (1.095 g) sodium methoxide was added causing the suspension to turn into a solution that was concentrated on a rotavapor. The solution turned into a gel when ˜150 ml remained. Upon continued evaporation the gel turned into a powder. The solids were stirred with 950 ml 96% EtOH for an hour and filtered. This gave 9 g of material after drying. The filtrate gave after evaporation and drying 2.9 g material. As HPLC showed that the material from the mother liquor contained less of the undesired diastereomer than the 9 g portion (0.25% versus 0.85) the materials were pooled and reslurried in 950 ml 96% ethanol for 2 hours before filtration. The mother liquor (that contained most of the product) was evaporated and re-slurried in 170 ml abs. ethanol. Filtration and wash with 100 ml abs. ethanol gave a gel-like cake that was vacuum dried yielding 8.1 g of 8-Br-PET-cGMP sodium salt with 0.5% undesired diasteromer (97% NMR assay, 72% yield).

### Example 2—Chromatography-Free Synthesis of a Diastereomerically Pure Rp-Guanosine-3′, 5′-Cyclic Phosphorothioate Analogues

### 2.1 Summary

Cyclic guanosinemonophosphorothioate analogue 1a is currently showing potential as a drug for the treatment of inherited retinal neurodegenerations. To support its ongoing preclinical development, we have developed a diastereoselective and chromatography-free synthesis for its preparation. Notable features in the synthetic sequence include a silylation step with 80% selectivity for the 2′, 5′- over the 3′, 5′-hydroxyls and a ring-closure of a 5′-H-phosphonate monoester with 90% selectivity for the S_{P}-diastereomer. Compounds were isolated via crystallization, including the final product 1a which was obtained as a Et₃NH⁺ salt in 125 g yield and >99.9% HPLC purity.

### 2.2 Introduction

Recent research suggests that photoreceptor death in several animal models for inherited retinal neurodegenerations (IRDs) is predominantly governed by an alternative non-apoptotic pathway which is partly mediated by over-activation of guanosine-dependent protein kinases (PKG). This over-activation stems from an unnatural build-up of cyclic guanosinemonophosphate (cGMP) in photoreceptors caused by disruptions of the phototransduction cascade. This sparked an interest in exploring cGMP analogues that could block this PKG-mediated pathway and thus potentially act as therapeutic agents for IRDs. The result was the discovery of a promising cyclic guanosinemonophosphorothioate (cGMPS) analogue, 1a, with potent neuroprotective effects in vivo. The compound is an 8-bromo-cGMPS derivative with a phenylethenyl (PET) group on the nucleobase and an R_{P}-configuration on the thiophosphate. Cyclic nucleotidemonophosphorothioates (cNMPSs) are known to resist cleavage by phosphodiesterases and therefore survive longer in cells than their phosphate counterparts, which adds to the potential of 1a as a therapeutic agent. In contrast to its antagonistic activity towards PKG, its Sp-diastereomer 1b and their phosphate equivalent 1c were both found to be PKG agonists similarly to natural cGMP (Scheme 1).

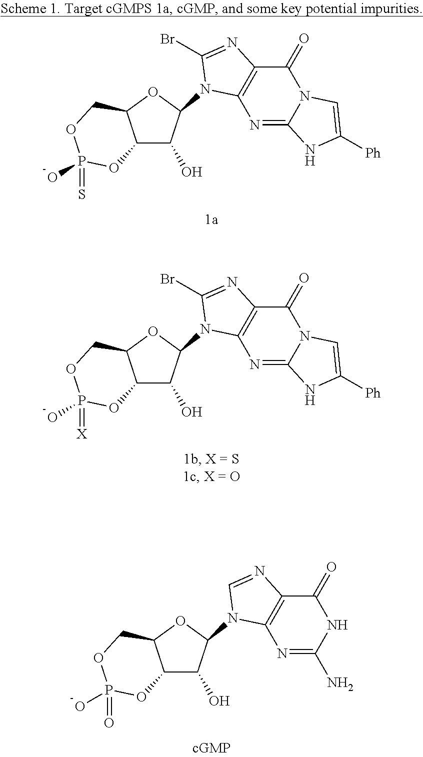

To enable further pre-clinical development, a larger supply of this active pharmaceutical ingredient (API) is desired, and our work towards a scalable and robust synthetic process for the preparation of highly pure 1a is presented here. Published cyclic phosphorothioate syntheses include those by Stec et al. (Stereospecific Synthesis of Adenosine 3′, 5′-(Sp)- and -(Rp)-Cyclic Phosphorothioates (cAMPS). Journal of the Chemical Society, Chemical Communications 1979, 940-941), involving reactions of phosphorylchlorides with aniline to obtain a mixture of phosphoroanilidate diastereomers which are then separated. The desired isomer is treated with a strong base and carbon disulfide (Stec reaction) to yield the cyclic thiophosphate with complete retention of configuration. Eckstein and coworkers used bisnitrophenylphosphorochloridothioate on unprotected nucleosides to prepare 5′-bisnitrophenylphosphorothioates. These were directly cyclized by treatment with potassium tert-butoxide. Genieser's group used a similar approach by employing thiophosphorylchloride to obtain 5′-thiophosphorodichloridates as precursors for the cyclization step, and were in fact the first group to synthetize 1a as described in U.S. Pat. No. 5,625,056. In all the above cases, cNMPSs (or their precursors) were obtained as diastereomer mixtures later separated by chromatography. In a more recent synthesis, Andrei et al. (Organic & Biomolecular Chemistry 2007, 5, 2070-2080) took inspiration from these methods and developed a stereoselective chlorination-amidation-Stec sequence, this time with cyclic adenosinemonophosphate as the starting material. However, only the RP-diastereomer was available with this tactic, and further chromatographic purification was still required.

An entirely different approach towards cGMPSs is the H-phosphonate route developed by Stawinski's group (Org. Lett. 2013, 15, 4082-4085). It entails the internal cyclization of a nucleoside-3′-H-phosphonate monoester to form the corresponding 3′, 5′-cyclic H-phosphonate diester, followed by sulfurization to afford the cyclic thiophosphate. In their report it was shown that 3′, 5′-cyclic nucleoside-H-phosphonates were formed in a diastereostereoselective manner, and that this selectivity can be directed towards the diastereomer of preference.

### 2.3 Results and Discussion

Route Development. We found the latter approach to be most promising and thus it became the focus of our development. We noted that in the prior art the 1,N2-phenylethenyl (PET) group is introduced after formation of the cyclic phosphorothioate. Also, it was performed without any protection on the 2′-hydroxyl. A corresponding H-phosphonate approach would be more susceptible to side-reactions on the nucleobase and ribose. Therefore, our synthetic design aimed to introduce the PET group first as it should function analogously to a protecting group, and then introducing a protecting group at the 2′ in order to negate formation of 2′, 3′-cyclic byproducts. We also placed importance on avoiding chromatographic purifications in favor of crystallizations, as well as on the process' ability to control key impurities such as the undesired PKG agonists 1b and 1c.

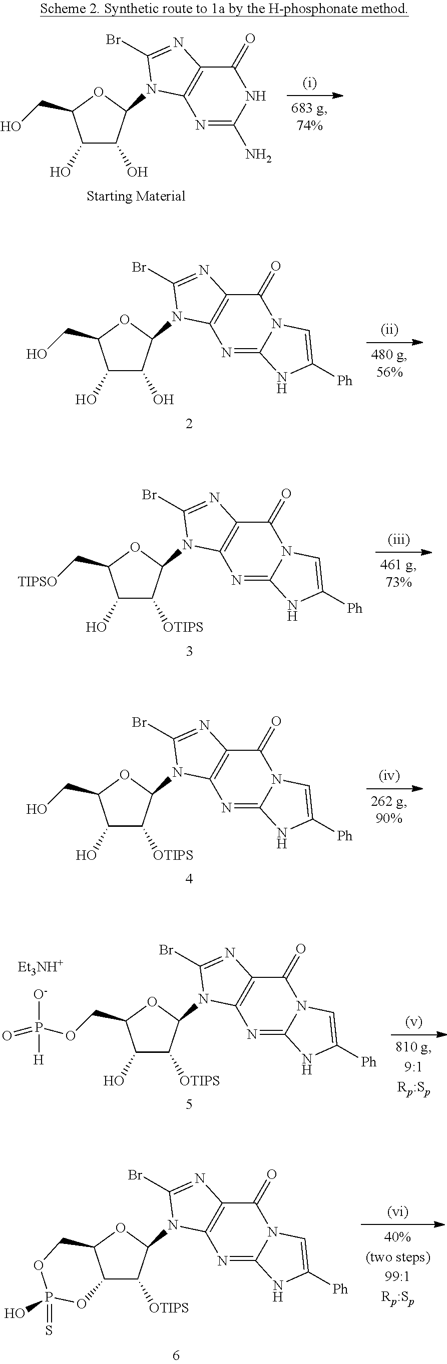

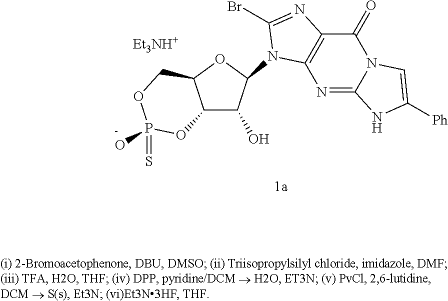 [SMILES: "Et3NH+" ; O=c1c2nc(Br)n([C@@H]3O[C@@H]4CO[P@@](O)(=S)O[C@H]4[C@H]3O)c2nc2[nH]c(-c3ccccc3)cn12]

Phenylethenylation. reported synthesis of an 8-bromoguanosine with a 1,N2-phenylethenyl (PET) modification used a 1,8-diazabicyclo[5.4.0]undec-7-ene (DBU)-DMSO combination and this served as starting point for our development. Dissolving 8-bromoguanosine in 10 volumes (ml/g) of DMSO and adding 5 equiv. of 2-bromoacetophenone followed by 7 equiv. of DBU caused consumption of starting material within 30 minutes with the formation of 8-bromoPETguanosine 2 as major product (˜70% LC area). Following neutralization using acetic acid, addition of water (15 volumes) precipitated the product, although as a slightly colored solid. It was also found that the amounts of both 2-bromoacetophenone and DBU could be reduced to 2.5 equiv. without compromising conversion. Overall, the reaction seemed to be robust as little variation in product formation was seen when varying stoichiometry. Next the volume efficiency and charging order was addressed. By adding DBU before the bromoketone the 8-bromoguanosine dissolved in only 4 volumes of DMSO. Adding the bromoacetophenone as a solution in one volume of DMSO to this mixture gave the product in around 50% yield. In addition, the 2-bromoacetophenone/DMSO solution was not stable, as shown by a steady yellowing of the clear solution as well as a build-up of byproducts on HPLC. This degradation does not seem to be related to the reduced yield, as adding the bromoacetophenone as a solid to a solution of the bromoguanosine and DBU in 5 volumes of DMSO gave no improvements. It was eventually found that vacuum drying the 8-bromoguanosine increased its solubility in DMSO. The commercial 8-bromoguanosine was found to contain ˜8% water (Karl Fischer), and this could be reduced to >0.5% by vacuum drying at temperatures up to 80° C. without affecting purity. Dried material gave clear solutions in 5 volumes of DMSO within 15 minutes. Regarding precipitation of the product, no improvement in purity or yields beyond 4 volumes of water (when using 5 volumes of DMSO) was found. Rapid water addition should be avoided as it caused sticky lumps to form. Interestingly, the same effect was obtained when the reaction mixture was not neutralized with acetic acid beforehand, even with slow addition of water. It was also found that a final cake wash using MeCN removed the color and some minor impurities. Thus, performing the reaction in 5 volumes of DMSO and precipitating the product using four volumes of water followed by a wash with MeCN yielded intermediate 2 as a white solid in 71% yield. The product was found to be crystalline and >98% pure (HPLC).

Protection strategy. Introducing a protecting group at the 2′-position cannot be achieved without also introducing one at the 5′, which then needs to be removed. Additionally, achieving selectivity for the 2′-hydroxyl over the 3′-one is still a challenge due to the similar reactivities of the two. Usually, this is addressed by separation of isomers via chromatography, which we aim to avoid. It is possible to bypass this competition with the help of silylating agents that simultaneously block the 5′ and 3′-positions, which can be removed after further protection of the 2′-position, for instance 1,3-dihalo-1,1,3,3-tetraalkyldisiloxanes. Although this is a popular approach, it generally involves an extra step for the 2′-protection and we found better success by optimizing the traditional silylation for selectivity towards the 2′ and developing a selective crystallization as described below.

Selective protection. We began by exploring the most common silylating methods where a 1:2 silylating agent:base mixture, most commonly 2.5 equiv of t-butyldimethylsilyl chloride (TBDMS) and 5 equiv. of imidazole. With this method, disilylation was achieved within 1.5 hours, but selectivity towards any of the disilylated isomers was virtually non-existent. Furthermore, the 2′, 3′, 5′-trisilylated byproduct constituted 44% of the product composition. When we replaced TBDMS-Cl with TIPS—Cl, substitution at the second hydroxyl resulted in an 80:20 ratio favoring the 2′, and less than 1% trisubstitution was seen after three days. However, a 2′->3′ silyl migration does occur over time, and the initial 80:20 isomer ratio degraded over several days to reach 60:40 at equilibrium. Unfortunately, protection of the second hydroxyl position was slow enough to allow the mixture to undergo some isomerization before complete conversion. In one trial using 130 g of starting material, only 1.5% 5′-O-TIPS—8-bromoguanosine remained after two days and the disilylated products had a 70:30 isomer ratio. Following an extractive work-up and evaporation, the resulting oil was subjected to crystallization studies. It was found that crystallization could be induced from several solvents, although several days could be required for complete desaturation. MeOH was found to selectively crystallize the 3′, 5′ isomer, while other solvents were selective for the 2′, 5′ isomer. Although, for the crystallizations of the 2′, 5′-isomer only the isomer excess crystallized, leaving nearly 1:1 isomer mixtures in the mother liquor. As result, >98% pure crystalline material in 33% yield was obtained. Encouraged by this, we made attempts to improve on the product loss to filtrates. All the attempts to increase crystallization yields by changing solvent volumes and addition of antisolvents were unfruitful as increase in yield was always at the expense of purity. Next, a two-step crystallization was attempted, first removing the undesired isomer with a crystallization from MeOH and, after evaporation, recrystallizing the mother liquor residue from isopropyl acetate. This gave the desired isomer in a somewhat improved yield of 40%. Still, this two-step crystallization was time-consuming and therefore not adopted on scale. A few attempts to find dynamic conditions (addition of DBU and TEA tried) where the 3,′5′-isomer would isomerize to the desired 2′, 5′-during the crystallization failed. Either no isomerization or no crystallization occurred. The conclusion from the crystallization studies was that to increase the isolated yield of 3, the regioselectivity of the silylation had to be improved. It proved to be a challenge to increase the silylation rate without equally increasing 2′->3′ silyl migration rates. Attempts included other solvents (pyridine, NMP tried), using various bases, and increasing reaction temperatures. Extensive byproduct formation was observed on the latter, and while some conditions were found which improved isomer ratios at full conversion (See entry 3 in Table 1) the most impressive result came from doubling the amount of TIPS—Cl (Entry 4, Table 1). This encouraged the second silylation step to complete after one night without significant isomerization. Finally, crystallization of this isomer mixture yielded 3 in 58% yield.

TABLE 1
Effect of reaction conditions on disilylation rate
in terms of monosilyl intermediate consumed, and the
resulting ratio of 2′:3′-diprotected nucleosides.

| | TIPS-CI | Base | | Monosilyl | Disilyl |
|---|---|---|---|---|---|
| Entry | equiv. | equiv. | Time | (%) | Ratio (2′:3′) |
| 1 | 2.5 | 5 | 2 h | 85 | 85:15 |
| | | | Overnight | 20 | 75:25 |
| | | | 2 nights | 9 | 68:32 |
| 2^{a} | 2.5 | 5 | 2 h | 50 | 69:31 |
| | | | Overnight | 11 | 61:39 |
| 3 | 2.5 | 10 | 2 h | 85 | 83:17 |
| | | | Overnight | 27 | 66:34 |
| 4 | 5 | 5 | 2 h | 66 | 86:14 |
| | | | Overnight | 5 | 86:14 |
| 5 | 2.5 | 2.5 | 2 h | 89 | 84:16 |
| | | | Overnight | 44 | 81:19 |
| 6 | 2.5 | 5^{b} | 2 h | 14 | 69:31 |
| | | | Overnight | 9 | 69:31 |
| 7 | 2.5 | 5^{c} | 2 h | 66 | 70:30 |
| | | | Overnight | 34 | 69:31 |

Unless otherwise stated, Imidazole was used as the base.
^{a}Reaction run at 90° C., extensive byproduct formation observed;
^{b}DBU used as base;
^{c}Et₃N used as base.

Having this step as the first in the synthesis was also explored since it gave the lowest yields. However, crystallization of the resulting crude was unsuccessful and chromatography was needed for isolation. Moreover, the strongly basic conditions for the subsequent PET step also caused some silyl migration, and we did not pursue this sequence further.

Selective deprotection. We first investigated AcOH in H2O and TFA/H2 in THE. The starting material was insoluble in the first mixture, while the latter was found to give good results, with complete conversion overnight in mild conditions. Still, we explored the effects of solvents, acid strength and concentrations, and water content on the ratio of product 4 to over-hydrolyzed product 2 (see Table 2). Performing an alcoholysis with MeOH instead of a hydrolysis significantly slowed conversions, but adding water to this mixture somewhat restored reaction rates. Toluene, and DCM (CH₂Cl₂) gave slightly higher reaction rates and lower byproduct formation when compared to THE, even though two-phase systems were formed, and toluene was not able to dissolve the starting material as well as THE and DCM. DCM was favored as it also precipitates 2 continuously, facilitating its removal. Finally, despite the low solubility of the product in most solvents, only MeCN was able to crystallize it from the crude mixture, affording the 2′-monosilyloxy nucleoside 4 in 80% yield and >98% purity after three nights.

TABLE 2
Effect of reaction conditions on conversion of 2,′5′-disilylated
nucleoside 3 to 2′-monosilylated nucleoside
4 and over-hydrolyzed nucleoside 2.

| | Conditions | | | | | |
|---|---|---|---|---|---|---|
| | Solvent | Volumes of | | Compound (%) | | |
| Entry | (10 vol) | TFA/H₂O | Time | 3 | 4 | 2 |
| 1 | THF | 0.5/2 | 1 h | 49 | 51 | <1 |
| | | | overnight | <1 | 91 | 8 |
| 2 | | 0.5/1 | 1 h | 60 | 40 | <1 |
| | | | Overnight | 1 | 93 | 6 |
| 3 | | 0.5/0 | 1 h | 97 | 3 | <1 |
| | | | Overnight | 92 | 8 | <1 |
| 4 | | 0.1/2 | 1 h | 81 | 19 | <1 |
| | | | Overnight | 25 | 73 | 2 |
| 5 | DCM | 0.5/2 | 1 h | 43 | 57 | <1 |
| | | | Overnight | <1 | 98 | 2 |
| 6 | Toluene | 0.5/2 | 1 h | 25 | 73 | 2 |
| | | | Overnight | <1 | 98 | 2 |
| 7 | MeOH | 0.5/2 | 1 h | 89 | 11 | <1 |
| | | | Overnight | 32 | 63 | 4 |
| 8 | | 0.5/1 | 1 h | 92 | 7 | 1 |
| | | | Overnight | 37 | 59 | 4 |
| 9 | | 0.5/0 | 1 h | 98 | 2 | <1 |
| | | | overnight | 69 | 30 | 1 |

H-Phosphonate monoester formation. Diphenyl phosphite (DPP, diphenyl H-phosphonate) is a common reagent for the formation of nucleoside H-phosphonate monoesters. It readily undergoes transesterification with alcohols and nucleosides in pyridine forming mixed phenyl H-phosphonate diesters (e.g. diester 7, scheme 3). After hydrolysis of the phenyl moiety, a nucleoside monoester is obtained in good yield. When DPP (1 equiv. to reduce the risk of bis-ester formation since 4 has two free hydroxyls) was added to a solution of 4 in pyridine, several peaks appeared on ³¹P-NMR resonating between 0 and 15 ppm, corresponding to formation of the 3′, 5′-cyclic H-phosphonate, 3′-mixed ester, or dinucleoside ester in addition to the desired intermediate 7. As the system seemed too reactive, a less basic solvent mixture containing 5% pyridine in DCM was evaluated. Using this system, a clean formation of mixed ester 7 was seen on ³¹ P-NMR. Due to slow conversion rates, we increased the amount of DPP to 3 equiv., which gave full conversion within one hour and without side-product formation. Addition of water and Et₃N after one hour hydrolyzed the mixed ester 7 and afforded the triethylammonium 5′-H-phosphonate monoester 5. Extractive work-up removed most of the residues from the excess DPP and the resulting crude was screened for crystallization solvents. A solvent swap to EtOAc monoester 5 crystallized in 75% yield and >95% purity.

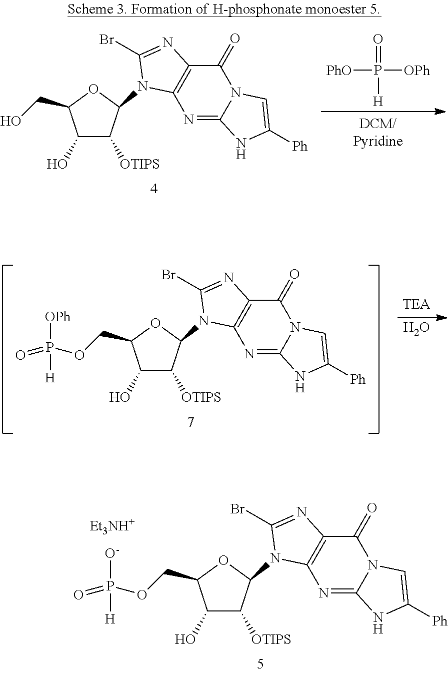

An alternative sequence was also briefly explored (scheme 4). Disilyl intermediate 3 could also be phosphonylated at the 3′-OH, yielding monoester 8. Acidic deprotection of the 5′-silyl group gave the 2′-protected-3′-H-phosphonate 9. We did not pursue this sequence further as initial crystallization attempts of intermediate 8 were not as successful as desired.

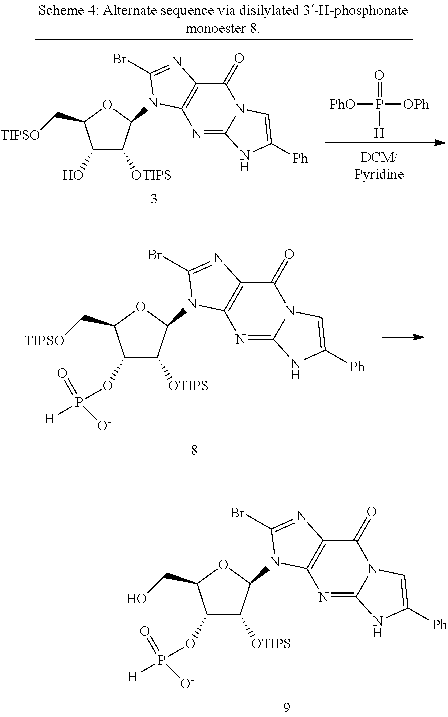

H-Phosphonate cyclization. We initially followed known conditions of DCM/Pyridine 19:1 mixtures using pivaloyl chloride (Pv-Cl) as coupling agent. It was observed within the first NMR spectrum recorded (after ca. 10 min) a 7:3 ratio between two peaks at 0.1 ppm (1JPH=734 Hz) and 5.8 ppm (1JPH=704 Hz) which is in accordance with the formation of the S_{P}:R_{P}-cyclic H-phosphonates. Treatment of this mixture with elemental sulfur after 20 min yielded the corresponding 7:3 R_{P}:S_{P} cyclic phosphorothioate mixture (Retention of configuration; 56.4 and 55.1 ppm, 3JPH=18 and 24 Hz respectively).

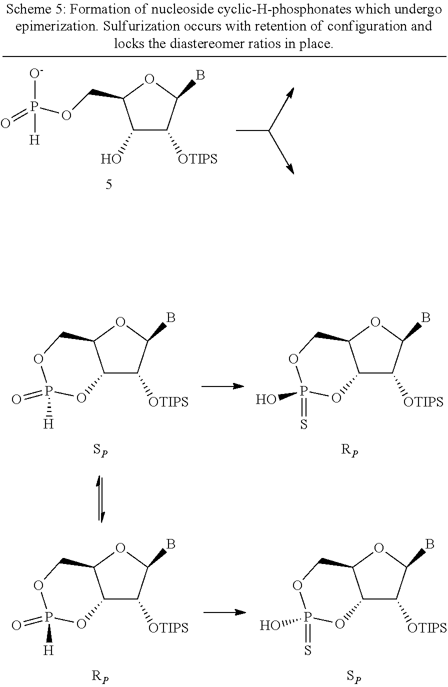

The isomerization behavior between the cyclic H-phosphonate diastereomers described by Stawinski's group was also observed: Allowing a ring-closure to stand overnight yielded the cyclic H-phosphonates in 1:9 S_{P}:R_{P} ratio (which would yield the sulfurized products in an undesired 1:9 R_{P}:S_{P} ratio), showing that the initially formed kinetic product is the desired cyclic Sp-H-phosphonate and that equilibrium favors the undesired diastereomer. To preserve the desired kinetic product, we explored 2,6-lutidine and DCM as both have been shown to reduce the rate of epimerization in contrast to pyridine. Performing the ring-closure in DCM in the presence of 5 equiv. of 2,6-lutidine gave the cyclic H-phosphonate in an initial 19:1 ratio in favor of the desired diastereomer (after ca. 10 min). The reaction was slower in this system and required ca. 15 min to reach completion, but this was counterbalanced by a highly suppressed epimerization evidenced by the 15:1 S_{P}:R_{P} ratio still seen for the cyclic H-phosphonate after standing for 1 hour. Sulfurizing the reaction after 1 hour gave the product in a ˜10:1 ratio in favor of the desired diastereomer. Usually, the final sulfurized mixture contained 75-80% of the desired product (according to ³¹ P-NMR), the remainder being the undesired diastereomer and small amounts of impurities in the phosphite area (>110 ppm) and product area (˜50 ppm), as well as some small amounts of starting material. Another commonly used coupling agent, diphenylchlorophosphate (DPCP), was also tried but it gave the same diastereomer ratios and offered no benefits. Large excess of coupling agent should be avoided as some side products became more pronounced, particularly with DPCP. Using 1.3 equiv. of Pv-Cl was found to be sufficient for full conversion as well as giving a reaction tolerant to adventitious water.

When sulfurizing the S_{P}:R_{P}—H-phosphonate, we found that a 50% excess of sulfur and Et₃N was sufficient. No other sulfurizing agents besides elemental sulfur were evaluated. Suitable sulfurization timings were determined by adding reaction aliquots to vials containing sulfur and Et₃N at different times after addition of Pv-Cl. Charging of sulfur before 45 minutes left unreacted intermediates, and longer times were associated with degrading diastereomeric ratios. However, after addition of sulfur/Et₃N the reaction mixtures were stable over several nights. After extractive work-up and a solvent swap to acetonitrile to precipitate residual sulfur, filtration and evaporation gave the crude product as a viscous oil. Despite several solvent screens, crystallization of the product as the triethylammonium salt from this crude were unsuccessful. We were able to force precipitation of the product by addition of 1 M HBr(aq) as an impure and amorphous solid which was easier to handle.

Deprotection of 2′-protected API. deprotection using Et₃N·3HF in MeCN, THF, dioxane and EtOH as solvent proved fruitful. The 2′-silyl was removed with no observable side-reactions, and we were delighted to find the target cGMPS 1a continuously precipitated out over the course of three days and the mother liquor enriched with the undesired diastereomer and the other impurities. We chose to pursue THE as the solvent as it gave the lowest losses to the mother liquor. The crude product was a slightly yellow crystalline salt that was almost pure on LC. However, its ¹H spectrum revealed the presence of two equivalents of triethylammonium, despite vacuum drying. ¹⁹F NMR showed a broad peak around −162 ppm showing the presence of fluoride complex, presumably triethylammonium fluoride. A reslurry in MeCN removed the fluoride, excess Et₃N, as well as all color, yielding the product in crude form (˜1% undesired diastereomer). Ethanol was found to be a suitable recrystallization solvent from which the product was recovered as a crystalline hemi-ethanol solvate. This solvate was stable and remained intact despite vacuum-drying, and calorimetry analyses showed no endotherms or loss of sample mass when heating. The crude product was found to be soluble in 20 volumes of boiling EtOH with no evident degradation, which made it possible to perform a cooling recrystallization. This afforded the target compound as a crystalline white powder with >99.9% LC purity. Confirmation of the correct structure and conformation came from comparison of its spectroscopic data with that of material previously prepared.

### 2.4 Experimental

8-Bromo-β-phenyl-1,N2-ethenoguanosine (2). 8-Bromoguanosine (683 g, 1.86 mol) was dissolved in DMSO (3.41 L, 5 vol.) and DBU (706 g, 2.5 eq., 4.64 mol). 2-Bromoacetophenone (443 g, 1.5 eq., 2.23 mol) was charged dropwise over 30 minutes to the reaction mixture which was kept at around 25° C. The reaction was stirred for 1 h before addition of conc. acetic acid (334 g, 3 eq., 5.57 mol). The product was precipitated by slow addition of water (2.7 L, 4 vol.) to the reaction mixture, filtered, and washed with water (5.5 L, 8 vol.), followed by MeCN (5.5 L, 8 vol.). After drying under a vacuum at 80° C. for 5 h, the nucleoside 2 (613 g, 71.5%) was recovered as a white crystalline solid. MS (M-H) m/z: 460.0257, found: 460.0259 (ES−).

8-Bromo-β-phenyl-1,N2-etheno-2′, 5′-ditriisopropylsilyloxyguanosine (3). The starting nucleoside 2 (480 g, 0.99 mol) was suspended in DMF (2.4 L, 5 vol.). Imidazole (336 g, 5 eq., 4.93 mol) was added to the suspension, followed by TIPS—Cl (951 g, 5 eq., 0.54 mmol), which caused the starting material to gradually dissolve. The mixture was stirred overnight before quenching with water (178 mL, 10 eq., 9.87 mol), and later diluted with toluene (7.2 L, 15 vol.). The organic phase was washed with water (3×2.4 L) and evaporated. i-PrOAc (2.4 L, 5 vol.) was added to the resulting crude oil and stirred overnight. The solids were filtered, washed with one cake-volume (672 mL) of i-PrOAc, and vacuum-dried at 35° C. overnight, affording the diprotected nucleoside 3 (462.4 g, 58.4%) as a white crystalline solid. MS (M-H) m/z: 772.2925, found: 772.2950 (ES−).

8-Bromo-β-phenyl-1,N2-etheno-2′-triisopropylsilyloxyguanosine (4). 2′, 5′-Disilylated nucleoside 3 (461 g, 0.57 mol), was dissolved in DCM (4.61 L, 10 vol.). TFA (231 mL, 0.5 vol.) and water (922 mL, 2 vol.) were charged into the vessel and stirred overnight. The mixture was neutralized with 35% aq. ammonia (0.25 vol.), filtered through packed celite to remove over-hydrolyzed byproduct, and washed with DCM (922 mL, 2 vol.). The organic phases were washed with water (3×100 mL) and evaporated, affording a crude solid which was resuspended in MeCN (3.23 L, 7 vol.) overnight. Filtering and washing the solids with MeCN (461 mL, 1 vol.), followed by vacuum-drying at 35° C. overnight gave the monoprotected nucleoside 4 (257 g, 74.2%) as a white crystalline solid. MS (M-H) m/z: 616.1591, found: 616.1615 (ES−).

Triethylammonium 8-Bromo-β-phenyl-1,N2-etheno-2′-triisopropylsilyloxyguanosine-5′-H-phosphonate (5). Compound 4 (262 g, 0.42 mol) was dissolved in DCM (4.98 L, 19 vol.) and pyridine (262 mL, 1 vol.). Diphenylphosphite (292 g, 3 eq., 1.25 mol) was charged and the reaction mixture. After 2 h the reaction was quenched with water (262 mL, 1 vol.) and Et₃N (262 mL, 1 vol.) and stirred for 1 h. The mixture was washed with water (2×2.5 L) and the organic phase was co-evaporated with 2-propanol and subsequently with ethyl acetate. The crude was recrystallized from ethyl acetate (2.62 L, 10 vol.) and the solids were filtered and washed with ethyl acetate (524 mL, 2 vol.) giving the monoester5 (312 g, 90.0%) as a white crystalline powder. MS (M-Et₃NH+) m/z: 680.1305, found: 680.1315 (ES−).

Rp-8-Bromo-β-phenyl-1,N2-etheno-2′-triisopropylsilyloxyguanosine-3′, 5′-cyclic monophosphorothiotic acid (6). 2,6-Lutidine (199 g, 5 eq., 1.86 mol) was added to a solution of 5 (310 g, 0.37 mol) in DCM (6.2 L, 20 vol.), followed by pivaloyl chloride (67 mL, 1.5 eq., 0.56 mol). After stirring for 1 hour, sulfur (18 g, 1.5 eq., 0.56 mol) and subsequently triethylamine (56 g, 1.5 eq., 0.56 mol) were charged to the vessel. After one hour, the solution was washed with water (2×1.24 L) and the organic phase evaporated. The residues were stirred in MeCN (1.55 L, 5 vol.), which precipitated sulfur as yellow crystals that were filtered out. Hydrobromic acid (3.1 L, 10 vol., 1 M) was added to the resulting filtrate precipitating a crude solid which was filtered out and resuspended in MeCN (1.55 L, 5 vol.), filtered, and washed with one cake-volume of MeCN. Removal of solvent residues gave the phosphorothioate 6 as a crude white solid which was used for the following step. MS (M-H) m/z: 694.0920, found: 694.0978 (ES−).

Triethylammonium Rp-8-Bromo-β-phenyl-1,N2-ethenoguanosine-3′, 5′-cyclic monophosphorothioate (1a). Triethylamine trishydrofluoride (620 mL, 2 vol.) was charged to a solution of the crude phosphorothiotic acid 6 in THE (1.24 L, 4 vol.). The mixture was seeded and stirred which caused a precipitate to form over three nights. The solids were filtered out and washed with THE (500 mL, 1.6 vol.), then resuspended in MeCN (930 mL, 3 vol.) for 1 h before filtering and washing with one cake-volume of MeCN, affording crystalline 1a. A cooling recrystallization of this material from 20 volumes of 99% EtOH, followed by filtration and wash with one cake-volume of the same, affords the target cGMPS 1a (126.5 g, 49.5%, two steps) as a white crystalline powder with >99.9% purity. MS (M-Et₃NH+) m/z: 537.9586, found: 537.9581 (ES−).

### 2.5 Conclusion

A six-step batch process for preparation of cGMPS 1a using no chromatography or chiral auxiliaries was developed and upscaled. Use of the H-phosphonate approach allowed us to form the desired R_{P}-cyclic phosphorothioate in a 9:1 ratio to the undesired S_{P}-diastereomer 1b. A silyl protection strategy on the ribose was devised which could be optimized to give the desired 2′, 5′-disilylated nucleoside in an impressive 86:14 ratio to the 3′, 5′-isomer. Selective crystallizations were possible for all key steps. The process afforded a total of 126 grams of >99.9% pure, crystalline 1a (13.8% total yield).

1. Method for producing a cyclic guanosine-3′, 5′-monophosphate (cGMP) analogue or a synthetic intermediate thereof, the method comprising the steps of:
i) providing a guanosine analogue of general formula (I) or a salt thereof: 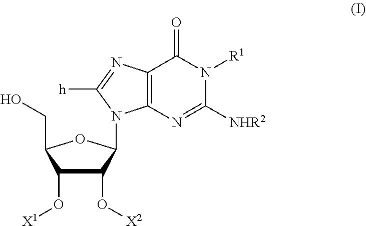
wherein:
h is H, halogen, or Q;
X¹ and X² are each independently chosen from H or p′;
p′ is in each instance independently chosen from a hydroxyl protective group;
R¹ and R² are each independently chosen from H, —(CH₂)ₙ—H, —(CH₂)ₙ—C₃₋₉heterocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—;
ar is in each instance independently a 5- or 6-membered aromatic or heteroaromatic ring, p wherein each instance of ar is individually optionally substituted with halogen, —OH, —SH, —NH₂, —NO₂, —OCH₃, —CH₃, —CH₂CH₃, —CH(CH₃)₂, or —CF₃, and is optionally fused with a second instance of ar;
Q is —(CH₂)ₙ—S—(CH₂)ₙ—H, —S—(CH₂)˜—OH, —S—(CH₂)ₙ—NH₂, —(CH₂)˜—O—(CH₂)ₙ—H, —O—(CH₂)˜—OH, —O—(CH₂)ₙ—NH₂, —O—C(CH₃)₃, —O—CH(CH₃)₂, —(CH₂)ₙ—N(—[CH₂]ₙH)₂, —NH—(CH₂)ₙNH₂, —NH—(CH₂)˜—OH, —(CH₂)ₙ—Nc¹c² wherein c¹ and c² together with the N to which they are attached form a 3 to 8 membered heterocycle or wherein c¹ is H and c² is a 3 to 8 membered heterocycle, —(CH₂)ₙ—H, —N₃, —CF₃, —(CH₂)ₙ-ar, —O—(CH₂)ₙ-(ar), —NH—(CH₂)ₙ-(ar), —S—(CH₂)ₙ-(ar), —(CH₂)ₙ-amido-ar, —O—(CH₂)ₙ-amido-(ar), —NH—(CH₂)ₙ-amido-(ar), —S—(CH₂)ₙ-amido-(ar), or a linker moiety, wherein any —H may be optionally replaced by a halogen, wherein each instance of n is independently chosen from 0, 1, 2, 3, 4, 5, 6, 7, or 8;
ii) contacting the provided guanosine analogue with a phosphorous oxoacid derivative to obtain a guanosine 5′-monophosphorous oxoacid ester analogue; and
iii) isolating the obtained guanosine 5′-monophosphorous oxoacid ester analogue by crystallization.

2. The method according to claim 1, wherein for the guanosine analogue of general formula (I) or salt thereof used in step i):
h is H or halogen or Q;
X¹ is H and X² is p′;
p′ is selected from the group consisting of methoxymethyl (MOM), tetrahydropyranyl (THP), t-butyl (tBu), allyl (all), benzyl (Bn), (tri)alkylsilyl (such as t-butyldimethylsilyl (TBDMS), triisopropylsilyl (TIPS), or t-butyldiphenylsilyl (TBDPS)), acyl (such as acetyl (Ac), pivaloyl (Pv), or benzoyl (Bz)), preferably from THP, (tri)alkylsilyl, and acyl;
R¹ and R² together form —CH═C(ar)-;
ar is phenyl, 4-methylphenyl, 3-thiophenyl, or 2-furanyl; and/or
Q is furanyl, —CF₃, —SCH₃, —S(isopropylphenyl), —S(phenylamidomethyl), —S(halophenyl), —S(hydroxyphenyl), —S(aminophenyl), —S(nitrophenyl), —S(methoxyphenyl), —S(toluyl), —S(trifluoromethylphenyl), —Nc¹c² wherein ^{c} and c² together with the N to which they are attached form a 3 to 8 membered heterocycle, —S—(CH₂)ₙ—OH, —S—(CH₂)ₙ—NH₂, —NH—(CH₂)ₙNH₂, or —NH—(CH₂)ₙOH, preferably furanyl, —CF₃, —S(4-hydroxyphenyl), or —S(4-chlorophenyl).

3. The method according to claim 1, wherein for the guanosine analogue of general formula (I) or salt thereof used in step i):
h is Br;
X¹ is H and X² is p′;
p′ is triisopropylsilyl (TIPS);
R¹ and R² together form —CH═C(ar)-; and
ar is phenyl.

4. The method according to claim 1, wherein the phosphorous oxoacid derivative of step ii) is a phosphorylating agent or a phosphonylating agent.

5. The method according to claim 1, wherein
the phosphorous oxoacid derivative of step ii) is of general formula (P): 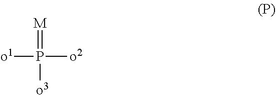
wherein:
M is S or O or is absent;
o¹ and o² are each independently selected from halogen, —O—C₁₋₈hydrocarbon, —S—C₁₋₈hydrocarbon, —NH—C₁₋₈hydrocarbon, borano, methylborano, dimethylborano, cyanoborano, and —N(C₁₋₈hydrocarbon)₂; and
o³ is H or is as defined for o¹; or of and o³ together form a chiral auxiliary that is preferably a C₂₋₁₂hydrocarbon.

6. The method according to claim 1, wherein the guanosine 5′-monophosphorous oxoacid ester analogue obtained in step ii) is of general formula (II) or a salt thereof: 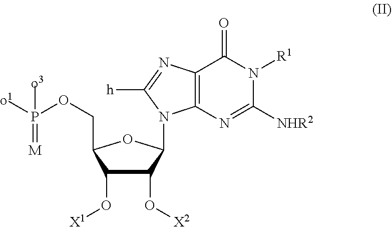
wherein h, X¹, X², R¹, and R²;
h is H, halogen, or O;
X¹ and X² are each independently chosen from H or in each instance independently chosen from a hydroxyl protective group;
R¹ and R² are each independently chosen from H, —(CH₂)ₙ—H, —(CH₂)ₙ—C₃₋₉hetereocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—
wherein o¹ and o³ are each independently —OH or are each independently selected from halogen, —O—C₁₋₈hydrocarbon, —S—C₁₋₈hydrocarbon, —NH—C₁₋₈hydrocarbon, borano, methylborano, dimethylborano, cyanborano, and —N(C₁₋₈hydrocarbon)₂ or o³ is H; and
wherein M is S or O.

7. The method according to claim 1, wherein X¹ is H and X² is p′, wherein the guanosine analogue of general formula (I) or salt thereof is provided by the steps of:
Ia) providing an unprotected guanosine analogue of general formula (pI) or a salt thereof: 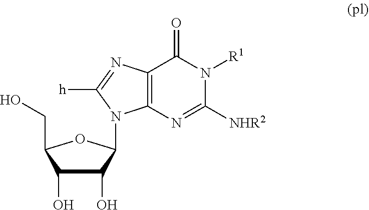
wherein
h is H, halogen, or O;
R¹ and R² are each independently chosen from H, —(CH₂)ₙ—H, —(CH₂)ₙ—C₃₋₉heterocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—;
Ib) contacting the unprotected guanosine analogue with (tri)alkylsilylhalide to obtain a multiply protected guanosine analogue and optionally isolating the multiply protected guanosine analogue by crystallization; and
Ic) selectively deprotecting the multiply protected guanosine analogue to obtain the guanosine analogue of general formula (I) wherein X¹ is H and X² is p′; and
Id) optionally isolating the obtained guanosine analogue of general formula (I) wherein X¹ is H and X² is p′ by crystallization.

8. The method according to claim 1, further comprising a step:
iv) cyclizing the guanosine 5′-monophosphorous oxoacid ester analogue obtained in step ii) to obtain a cyclic guanosine-3′, 5′-monophosphate (cGMP) analogue, wherein said cyclisation is preferably performed in the presence of a sterically hindered base.

9. The method according to claim 8 wherein the cGMP analogue is of general formula (III) or a salt thereof: 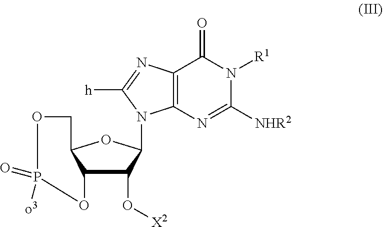
wherein
h is H, halogen, or O;
X² is H or a hydroxyl protective group;
R¹ and R² are each independently chosen from H, —(CH₂)ₙ—H, —(CH₂)ₙ—C₃₋₉hetereocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—
wherein o³ is —OH, H, halogen, —O—C₁₋₈hydrocarbon, —S—C₁₋₈hydrocarbon, —NH—C₁₋₈hydrocarbon, borano, methylborano, dimethylborano, cyanborano, and —N(C₁₋₈hydrocarbon)₂;
the method optionally further comprising a step:
v) contacting the cGMP analogue with a sulfurizing agent to obtain a thiolated cGMP analogue of general formula (III) wherein o³ is —SH or —S—C₁₋₁₂hydrocarbon.

10. The method according to claim 9, wherein the cGMP analogue of general formula (III) is of general formula (III-Rp): 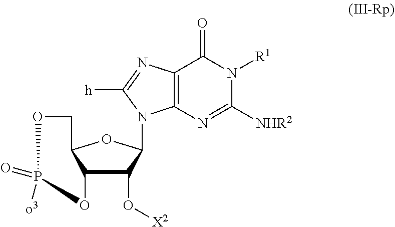
wherein
h is H, halogen, or O;
X² is H or a hydroxyl protective group;
R¹ and R² are each independently chosen from H, —(CH₂)ₙ—H, —(CH₂)ₙ—C₃₋₉hetereocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—;
wherein o³ is —OH, H, halogen, —O—C₁₋₈hydrocarbon, —S—C₁₋₈hydrocarbon, —NH—C₁₋₈hydrocarbon, borano, methylborano, dimethylborano, cyanborano, and —N(C₁₋₈hydrocarbon)₂.

11. The method according to claim 9 wherein X² is p′, further comprising the steps of
vi) deprotecting the hydroxyl moiety that is protected by X² to obtain a deprotected cGMP analogue; and
vii) optionally triturating the deprotected cGMP analogue; and
viii) optionally converting the deprotected cGMP analogue to a pharmaceutically acceptable salt.

12. A compound of general formula (II) or a salt thereof: 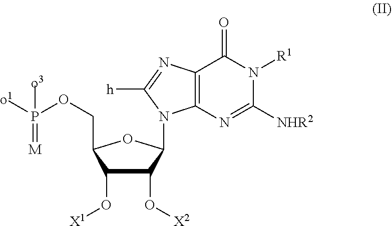
wherein
h is H, halogen, or O;
X¹ and X² are each independently chosen from H or in each instance independently chosen from a hydroxyl protective group;
R¹ and R² are each independently chosen from H, —(CH₂)ₙ—H, —(CH₂)ₙ—C₃₋₉hetereocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—
wherein o¹ and o³ are each independently —OH, -halogen, —O—C₁₋₈hydrocarbon, —S—C₁₋₈hydrocarbon, —NH—C₁₋₈hydrocarbon, borano, methylborano, dimethylborano, cyanborano, and —N(C₁₋₈hydrocarbon)₂;
wherein M is S or O.

13. The compound according to claim 12, wherein o³ is H.

14. The compound according to claim 12, wherein
h is Br;
X¹ is H and X² is p′;
p′ is preferably triisopropylsilyl (TIPS);
R¹ and R² together form —CH═C(ar)-;
ar is phenyl;
o¹ is OH;
o³ is H; and
M is S or O.

15. The compound according to claim 12, wherein
h is Br;
X¹ is H and X² is p′;
p′ is preferably triisopropylsilyl (TIPS);
R¹ and R² together form —CH═C(ar)-;
ar is 4-methylphenyl;
o¹ is OH;
o³ is H; and
M is S or O.

16. The compound according to claim 12, wherein the compound is crystalline.

17. The compound according to claim 13, wherein the compound is crystalline.

18. The compound according to claim 14, wherein the compound is crystalline.

19. The compound according to claim 15, wherein the compound is crystalline.

20. The compound according to claim 13, wherein the compound is not of general formula (II) wherein
h is H;
X¹ is and X² together form an acetonide protecting group;
R¹ is H and R² is H or —CH₃;
o¹ is OH;
o³ is H; and
M is O.
